(19) United States
(12) Patent Application Publication
(10) Pub. No.: US 2018/0193482 A1
(43) Pub. Date: Jul. 12, 2018

(54) PATTERNED DOSING OF IMMUNOSUPPRESSANTS COUPLED TO SYNTHETIC NANOCARRIERS

(71) Applicants: Petr Ilyinskii, Cambridge, MA (US); Selecta Biosciences, Inc., Watertown, MA (US)

(72) Inventors: Petr Ilyinskii, Cambridge, MA (US); Takashi Kei Kishimoto, Lexington, MA (US)

(73) Assignee: Selecta Biosciences, Inc., Watertown, MA (US)

(21) Appl. No.: 15/863,076

(22) Filed: Jan. 5, 2018

Related U.S. Application Data
(60) Provisional application No. 62/545,412, filed on Aug. 14, 2017.
(60) Provisional application No. 62/445,637, filed on Jan. 12, 2017.
(60) Provisional application No. 62/443,658, filed on Jan. 7, 2017.

(51) Int. Cl. A61K 47/69; C12N 15/86; G16H 20/17; A61P 37/06

(52) U.S. Cl. CPC A61K 47/6901; A61K 47/6937; C12N 15/86; A61K 47/6907; A61P 37/06; C12N 2710/10042; A61K 47/6911; G16H 20/17

(57) ABSTRACT

Provided herein are methods and related compositions for administering viral vectors and synthetic nanocarriers comprising an immunosuppressant. In some embodiments, the methods and compositions provided herein achieve improved transgene expression and/or immune response reduction, such as downregulated IgM and/or IgG immune responses.

### RELATED APPLICATIONS

This application claims the benefit of priority under 35 U.S.C. § 119 to U.S. Provisional Application No. 62/443,658 filed Jan. 7, 2017, U.S. Provisional Application No. 62/445,637 filed Jan. 12, 2017, and U.S. Provisional Application No. 62/545,412 filed Aug. 14, 2017, the entire contents of each of which are incorporated herein by reference.

### FIELD OF THE INVENTION

This invention relates, at least in part, to methods, and related compositions, for administering viral vectors and synthetic nanocarriers comprising an immunosuppressant. In some embodiments, the methods and compositions provided herein achieve increased transgene expression and/or reduced immune responses, such as downregulated IgM and/or IgG immune responses against the viral vectors.

### SUMMARY OF THE INVENTION

In one aspect, a method comprising coadministering a first round of viral vector and synthetic nanocarriers comprising an immunosuppressant to a subject, and administering synthetic nanocarriers comprising an immunosuppressant at one or more time points prior and/or subsequent to the first round coadministration, wherein the prior and/or subsequent administrations of the synthetic nanocarriers comprising an immunosuppressant occurs within 1 month, 2 weeks, 1 week, 1 day, 12 hours, 6 hours, 1 hour, 30 minutes, or 15 minutes, prior or subsequent to, respectively, the first round coadministration is provided.

In one embodiment of any one of the methods provided herein, the method further comprises coadministering a second round of viral vector and synthetic nanocarriers comprising an immunosuppressant to the subject, and administering synthetic nanocarriers comprising an immunosuppressant at one or more time points prior and/or subsequent to the second round coadministration, wherein the prior and/or subsequent administrations of the synthetic nanocarriers comprising an immunosuppressant occurs within 1 month, 2 weeks, 1 week, 1 day, 12 hours, 6 hours, 1 hour, 30 minutes, or 15 minutes, prior or subsequent to, respectively, the second round coadministration.

In one aspect, a method comprising coadministering synthetic nanocarriers comprising an immunosuppressant and a viral vector to a subject, and administering at least one pre-dose and/or at least one post-dose of the synthetic nanocarriers comprising an immunosuppressant without the viral vector to the subject is provided.

In one embodiment of any one of the methods provided, at least one pre-dose and at least one post-dose is administered to the subject. In one embodiment of any one of the methods provided, at least two pre-doses are administered to the subject. In one embodiment of any one of the methods provided, at least two post-doses are administered to the subject.

In one embodiment of any one of the methods provided, the coadministering is repeated in the subject.

In one embodiment of any one of the methods provided, at least one pre-dose and/or at least one post-dose of the synthetic nanocarriers comprising an immunosuppressant without the viral vector is administered to the subject with each repeated coadministering step. In one embodiment of any one of the methods provided, at least one pre-dose and at least one post-dose is administered to the subject with each repeated coadministering step. In one embodiment of any one of the methods provided, at least two pre-doses are administered to the subject with each repeated coadministering step. In one embodiment of any one of the methods provided, at least two post-doses are administered to the subject with each repeated coadministering step.

In one embodiment of any one of the methods provided, administration of the pre-dose(s) and/or post-dose(s) occurs within 1 month prior or subsequent to, respectively, a coadministration. In one embodiment of any one of the methods provided, administration of the pre-dose(s) and/or post-dose(s) occurs within 2 weeks prior or subsequent to, respectively, to a coadministration. In one embodiment of any one of the methods provided, administration of the pre-dose(s) and/or post-dose(s) occurs within 1 week prior or subsequent to, respectively, to a coadministration. In one embodiment of any one of the methods provided, administration of the pre-dose(s) and/or post-dose(s) occurs within 3 days prior or subsequent to, respectively, to a coadministration. In one embodiment of any one of the methods provided, administration of the pre-dose(s) and/or post-dose(s) occurs within 2 days prior or subsequent to, respectively, to a coadministration. In one embodiment of any one of the methods provided, administration of the pre-dose(s) and/or post-dose(s) occurs within 1 day prior or subsequent to, respectively, to a coadministration. In one embodiment of any one of the methods provided, administration of the pre-dose(s) and/or post-dose(s) occurs within 12 hours prior or subsequent to, respectively, to a coadministration. In one embodiment of any one of the methods provided, administration of the pre-dose(s) and/or post-dose(s) occurs within 6 hours prior or subsequent to, respectively, to a coadministration. In one embodiment of any one of the methods provided, administration of the pre-dose(s) and/or post-dose(s) occurs within 1 hour prior or subsequent to, respectively, to a coadministration. In one embodiment of any one of the methods provided, administration of the pre-dose(s) and/or post-dose(s) occurs within 30 minutes prior or subsequent to, respectively, to a coadministration. In one embodiment of any one of the methods provided, administration of the pre-dose(s) and/or post-dose(s) occurs within 15 minutes prior or subsequent to, respectively, to a coadministration.

In one embodiment of any one of the methods provided, each pre-dose and/or post-dose is administered within 3 days of the coadministering step. In one embodiment of any one of the methods provided, each pre-dose and/or post-dose is administered within 2 days of the coadministering step.

In one embodiment of any one of the methods provided, each post-dose is administered biweekly after the coadministering step.

In one embodiment of any one of the methods provided, the amount of the immunosuppressant of each pre-dose is the same as the amount of the immunosuppressant of each coadministering step. In one embodiment of any one of the methods provided, the amount of the immunosuppressant of each post-dose is the same as the amount of the immunosuppressant of each coadministering step.

In one embodiment of any one of the methods provided, each pre-dose, post-dose and/or coadministering step is by intravenous administration.

In one aspect, a method comprising to a first subject, (1) coadministering (a) a dose of immunosuppressant comprised in synthetic nanocarriers and (b) a dose of a viral vector, and (2) administering, without a dose of the viral vector, (c) a pre-dose and/or a post-dose of the immunosuppressant comprised in synthetic nanocarriers, wherein the amount of the immunosuppressant of (a) and (c) together is equal to an amount of immunosuppressant of (d) a dose of the immunosuppressant comprised in synthetic nanocarriers that when coadministered with the viral vector, without a pre-dose or a post-dose of the immunosuppressant coupled to synthetic nanocarriers, reduces an immune response against the viral vector or increases transgene expression of the viral vector in a second subject is provided.

In one embodiment of any one of the methods provided, the amount of the immunosuppressant of the pre-dose or post-dose of (c) is no more than half of the amount of (d). In one embodiment of any one of the methods provided, the amount of the immunosuppressant of the pre-dose or post-dose of (c) is half the amount of (d).

In one embodiment of any one of the methods provided, a pre-dose and a post-dose is administered to the first subject in (c). In one embodiment of any one of the methods provided, the amount of the immunosuppressant of the pre-dose and post-dose of (c) is the same. In one embodiment of any one of the methods provided, the amount of the immunosuppressant of (a) is the same as the amount of the pre-dose or post-dose of (c).

In one embodiment of any one of the methods provided, in (c) at least two pre-doses are administered to the first subject. In one embodiment of any one of the methods provided, in (c) at least two post-doses are administered to the first subject.

In one embodiment of any one of the methods provided, (1) and (2) are repeated.

In one embodiment of any one of the methods provided, administration of the pre-dose(s) and/or post-dose(s) occurs within 1 month prior or subsequent to, respectively, a coadministration. In one embodiment of any one of the methods provided, administration of the pre-dose(s) and/or post-dose(s) occurs within 2 weeks prior or subsequent to, respectively, to a coadministration. In one embodiment of any one of the methods provided, administration of the pre-dose(s) and/or post-dose(s) occurs within 1 week prior or subsequent to, respectively, to a coadministration. In one embodiment of any one of the methods provided, administration of the pre-dose(s) and/or post-dose(s) occurs within 3 days prior or subsequent to, respectively, to a coadministration. In one embodiment of any one of the methods provided, administration of the pre-dose(s) and/or post-dose(s) occurs within 2 days prior or subsequent to, respectively, to a coadministration. In one embodiment of any one of the methods provided, administration of the pre-dose(s) and/or post-dose(s) occurs within 1 day prior or subsequent to, respectively, to a coadministration. In one embodiment of any one of the methods provided, administration of the pre-dose(s) and/or post-dose(s) occurs within 12 hours prior or subsequent to, respectively, to a coadministration. In one embodiment of any one of the methods provided, administration of the pre-dose(s) and/or post-dose(s) occurs within 6 hours prior or subsequent to, respectively, to a coadministration. In one embodiment of any one of the methods provided, administration of the pre-dose(s) and/or post-dose(s) occurs within 1 hour prior or subsequent to, respectively, to a coadministration. In one embodiment of any one of the methods provided, administration of the pre-dose(s) and/or post-dose(s) occurs within 30 minutes prior or subsequent to, respectively, to a coadministration. In one embodiment of any one of the methods provided, administration of the pre-dose(s) and/or post-dose(s) occurs within 15 minutes prior or subsequent to, respectively, to a coadministration.

In one embodiment of any one of the methods provided, each pre-dose and/or post-dose is administered within 3 days of the coadministering step. In one embodiment of any one of the methods provided, each pre-dose and/or post-dose is administered within 2 days of the coadministering step. In one embodiment of any one of the methods provided, each post-dose is administered biweekly after the coadministering step.

In one embodiment of any one of the methods provided, each pre-dose, post-dose and/or coadministering step is by intravenous administration.

In one embodiment of any one of the methods provided, the viral vector comprises one or more expression control sequences. In one embodiment of any one of the methods provided, the one or more expression control sequences comprise a liver-specific promoter. In one embodiment of any one of the methods provided, the one or more expression control sequences comprise a constitutive promoter.

In one embodiment of any one of the methods provided, the method further comprises assessing an IgM and/or IgG response to the viral vector in the subject at one or more time points. In one embodiment of any one of the methods provided, at least one of the time points of assessing an IgM and/or IgG response is subsequent to a coadministration.

In one embodiment of any one of the methods provided, the viral vector and synthetic nanocarriers comprising an immunosuppressant are admixed for each coadministration.

In one embodiment of any one of the methods provided, the viral vector is a retroviral vector, an adenoviral vector, a lentiviral vector or an adeno-associated viral vector.

In one embodiment of any one of the methods provided, the viral vector is an adeno-associated viral vector. In one embodiment of any one of the methods provided, the adeno-associated viral vector is an AAV1, AAV2, AAV5, AAV6, AAV6.2, AAV7, AAV8, AAV9, AAV10 or AAV11 adeno-associated viral vector.

In one embodiment of any one of the methods provided, the immunosuppressant of the coadministration and/or pre-dose and/or post-dose is an inhibitor of the NF-kB pathway. In one embodiment of any one of the methods provided, the immunosuppressant of the coadministration and/or pre-dose and/or pos-dose is an mTOR inhibitor. In one embodiment of any one of the methods provided, the mTOR inhibitor is rapamycin.

In one embodiment of any one of the methods provided, the immunosuppressant is coupled to the synthetic nanocarriers. In one embodiment of any one of the methods provided, the immunosuppressant is encapsulated in the synthetic nanocarriers.

In one embodiment of any one of the methods provided, the synthetic nanocarriers of the coadministration and/or pre-dose and/or post-dose comprise lipid nanoparticles, polymeric nanoparticles, metallic nanoparticles, surfactant-based emulsions, dendrimers, buckyballs, nanowires, virus-like particles or peptide or protein particles.

In one embodiment of any one of the methods provided, the synthetic nanocarriers comprise polymeric nanoparticles. In one embodiment of any one of the methods provided, the polymeric nanoparticles comprise a polyester, polyester attached to a polyether, polyamino acid, polycarbonate, polyacetal, polyketal, polysaccharide, polyethyloxazoline or polyethyleneimine. In one embodiment of any one of the methods provided, the polymeric nanoparticles comprise a polyester or a polyester attached to a polyether. In one embodiment of any one of the methods provided, the polyester comprises a poly(lactic acid), poly(glycolic acid), poly(lactic-co-glycolic acid) or polycaprolactone. In one embodiment of any one of the methods provided, the polymeric nanoparticles comprise a polyester and a polyester attached to a polyether. In one embodiment of any one of the methods provided, the polyether comprises polyethylene glycol or polypropylene glycol.

In one embodiment of any one of the methods provided, the mean of a particle size distribution obtained using dynamic light scattering of a population of the synthetic nanocarriers is a diameter greater than 110 nm. In one embodiment of any one of the methods provided, the diameter is greater than 150 nm. In one embodiment of any one of the methods provided, the diameter is greater than 200 nm. In one embodiment of any one of the methods provided, the diameter is greater than 250 nm. In one embodiment of any one of the methods provided, the diameter is less than 5 μm. In one embodiment of any one of the methods provided, the diameter is less than 4 μm. In one embodiment of any one of the methods provided, the diameter is less than 3 μm. In one embodiment of any one of the methods provided, the diameter is less than 2 μm. In one embodiment of any one of the methods provided, the diameter is less than 1 μm. In one embodiment of any one of the methods provided, the diameter is less than 750 nm. In one embodiment of any one of the methods provided, the diameter is less than 500 nm. In one embodiment of any one of the methods provided, the diameter is less than 450 nm. In one embodiment of any one of the methods provided, the diameter is less than 400 nm. In one embodiment of any one of the methods provided, the diameter is less than 350 nm. In one embodiment of any one of the methods provided, the diameter is less than 300 nm.

In one embodiment of any one of the methods provided, the load of immunosuppressant comprised in the synthetic nanocarriers, on average across the synthetic nanocarriers, is between 0.1% and 50% (weight/weight). In one embodiment of any one of the methods provided, the load is between 0.1% and 25%. In one embodiment of any one of the methods provided, the load is between 1% and 25%. In one embodiment of any one of the methods provided, the load is between 2% and 25%.

In one embodiment of any one of the methods provided, an aspect ratio of a population of the synthetic nanocarriers is greater than 1:1, 1:1.2, 1:1.5, 1:2, 1:3, 1:5, 1:7 or 1:10.

In one aspect, a kit comprising one or more of any one of the pre-doses provided herein or one or more of any one of the post-doses provided herein, each, for example, as described in any one of the claims, and a dose of any one of the synthetic nanocarriers comprising an immunosuppressant provided herein for coadministration with a viral vector is provided.

In one embodiment of any one of the kits provided, the kit further comprises a dose of any one of the viral vectors provided herein.

In one embodiment of any one of the kits provided, the kit comprises one or more of any one of the pre-doses provided herein and one or more of any one of the post-doses provided herein.

In one embodiment of any one of the kits provided, the kit further comprises instructions for use. In one embodiment of any one of the kits provided, the instructions for use comprise instructions for performing any one of the methods provided herein.

In one embodiment of any one of the kits provided, the synthetic nanocarriers comprising an immunosuppressant for administration with a viral vector are any one of the synthetic nanocarriers comprising an immunosuppressant provided herein, for example, as described in any one of the claims.

In one embodiment of any one of the kits provided, the viral vector is any one of the viral vectors provided herein, for example, as described in any one of the claims.

In one embodiment of any one of the methods provided herein, the prior and/or subsequent administrations of the synthetic nanocarriers comprising an immunosuppressant do not include administration of the viral vector.

In another aspect, a kit comprising any one or combination of the synthetic nanocarriers of any one of the methods provided herein is provided. In one embodiment of any one of the kits provided, the kit further comprises the viral vector of any one of the methods provided herein. In one embodiment of any one of the kits provided, the kit further comprises one or more pre-doses and/or post-doses of any one of the methods provided herein.

### BRIEF DESCRIPTION OF THE FIGURES

FIGS. 1A and 1B show SEAP activity and AAV IgG antibody levels with and without synthetic nanocarriers comprising rapamycin.

FIGS. 2A and 2B show SEAP activity at d19 and d75, respectively. FIG. 2C shows AAV IgG antibody levels at both d19 and d75.

FIG. 3A shows SEAP expression dynamics. FIG. 3B shows AAV IgG antibody levels at d12 and d19.

FIGS. 4A and 4B show the size distribution by volume of AAV and synthetic nanocarriers comprising rapamycin.

FIG. 5A shows serum AAV IgM at d5 and d10 after AAV administration. FIG. 5B shows serum AAV IgM at d7, d12, d19 and d89.

FIG. 6 shows AAV IgM at d7 versus longitudinal AAV-driven SEAP expression.

FIG. 7 shows AAV IgG antibody levels at d7, d12, d19 and d33.

FIG. 8 shows SEAP expression dynamics (d7-d47).

FIG. 9 shows AAV IgM antibody levels at d5 and d13.

FIG. 10 shows AAV IgG antibody levels at d9, d13 and d20.

FIG. 11A is a graph showing SEAP expression dynamics at specific times following the initial AAV inoculation with AAV-SEAP ±synthetic nanocarriers comprising rapamycin (SVP[Rapa]). FIG. 11B is a graph showing AAV IgG formation at different time points following the initial AAV inoculation with AAV-SEAP ±synthetic nanocarriers comprising rapamycin (SVP[Rapa]).

FIG. 12 is a graph showing SEAP expression dynamics at specific times following injection with AAV-SEAP ±synthetic nanocarriers comprising rapamycin (SVP[Rapa]).

FIG. 13A is a graph showing AAV-driven SEAP expression dynamics at specific times in AAV8-pre-immunized mice. FIG. 13B is a graph showing AAV IgG formation at different time points with different combinations and regimens of SVP[Rapa] administration.

FIG. 14A is a graph showing SEAP expression dynamics in mice with a low AAV IgG and following two doses of synthetic nanocarriers comprising rapamycin (SVP[Rapa]). FIG. 14B is a graph showing SEAP expression at d139, comparing the group that received zero or one dose of synthetic nanocarriers comprising rapamycin (SVP[Rapa]) at AAV boost (d92) and the group that received two doses of synthetic nanocarriers comprising rapamycin (SVP[Rapa]) at AAV boost (d92). FIG. 14C is a graph showing AAV IgG dynamics after the AAV-(RFP/SEAP) administrations at the specified time points. FIG. 14D is a graph showing a negative correlation between AAV IgG and SEAP activity on d153.

FIG. 15A is a graph showing serum SEAP dynamics following the first AAV injection under different SVP[Rapa] administration regimens. FIG. 15B is a graph showing AAV IgG after AAV vector and synthetic nanocarriers comprising rapamycin (SVP[Rapa]) co-injection followed by different regimens of SVP[Rapa] administration.

FIG. 16 shows AAV IgG measurements on d116 in groups co-injected with AAV and SVP[Rapa] and then treated with different SVP[Rapa] regimens.

FIG. 17A is a graph that shows SEAP dynamics (AAV-SEAP, 1×1010 VG; d0/125) at different time points (days post-AAV priming dose). FIG. 17B is a graph depicting the results of an ELISA. The graphs show the levels of AAV IgG following different treatment regimens (on d7, d12, d19, d47 and d75).

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes. Such incorporation by reference is not intended to be an admission that any of the incorporated publications, patents and patent applications cited herein constitute prior art.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to “a DNA molecule” includes a mixture of two or more such DNA molecules or a plurality of such DNA molecules, reference to “an immunosuppressant” includes a mixture of two or more such immunosuppressant molecules or a plurality of such immunosuppressant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### A. Introduction

Viral vectors, such as those based on adeno-associated viruses (AAVs), have shown great potential in therapeutic applications, such as gene therapy. However, the use of viral vectors in gene therapy and other applications has been limited due to immunogenicity as a result of viral antigen exposure. Subjects exposed to viral vectors often display immune responses, and ultimately end up acquiring resistance to the viral vector and/or face significant inflammatory reactions. Both cellular and humoral immune responses against the viral vector can diminish efficacy and/or reduce the ability to use such therapeutics, such as in a repeat administration context. These immune responses include antibody, B cell, and T cell responses and can be specific to viral antigens of the viral vector, such as viral capsid or coat proteins, or peptides thereof.

The inventors have surprisingly discovered that dosing regimens that include a pre-dose and/or post-dose of synthetic nanocarriers comprising an immunosuppressant in combination with a coadministration of the synthetic nanocarriers and viral vector can achieve improved immune response reduction and/or improved transgene expression. Such improvements are significant as compared to coadministration of synthetic nanocarriers and viral vector alone (without a pre-dose or post-dose). For example, as shown in the Examples, it was demonstrated that additional administrations of rapamycin-comprising synthetic nanocarriers prior to and/or following the injection of liver-tropic AAV8 vector coadministered with the rapamycin-comprising synthetic nanocarriers, maintained the highest and the most stable levels of transgene expression after both initial and follow-up injections in naïve and AAV-immune animals. This combined with the lowest AAV antibody responses.

In addition, it was also surprisingly found that the amount of immunosuppressant, when comprised in synthetic nanocarriers, of a coadministration step could be reduced with a pre-dose or post-dose as compared to the coadministration step alone (without a pre-dose or post-dose). Thus, an amount of immunosuppressant, when comprised in synthetic nanocarriers, can be “split” amongst a pre-dose and/or post-dose and coadministered dose in any one of the treatment regimens provided herein. For example, the Examples demonstrate that splitting a dose of an immunosuppressant, when comprised in synthetic nanocarriers, into two parts and administering the first half dose prior to AAV vector co-injection with the second half dose was beneficial, both in terms of transgene expression and for suppressive effect on antiviral IgG, relative to when the same total dose of immunosuppressant, when comprised in synthetic nanocarriers, was simply co-injected with the AAV vector.

Additionally, it has been surprisingly discovered that viral vector administration can result in robust IgM immune responses shortly after viral vector administration. It has also been discovered that synthetic nanocarriers comprising an immunosuppressant and administered at times relative to the viral vector administration induce elevated transgene expression in an IgM-dependent manner, in some examples. Specifically, the synthetic nanocarriers were found to downregulate the induction of an IgM immune response to adeno-associated viral vectors and that early IgM levels were inversely correlated to transgene expression, with high IgM antibody levels following viral vector administration correlating to low levels of transgene expression, and vice versa. Further, this correlation was found to persist after an additional administration of a viral vector. Prior to these findings, it was shown that synthetic nanocarriers comprising immunosuppressants downregulate IgG antibody responses to a number of antigens, including soluble proteins and viral particles. However, for viral vector administration, it may be that other immune responses, such as IgM antibody responses, are as important, in certain contexts, such as, for example, transgene expression.

Therefore, the inventors have surprisingly and unexpectedly discovered that the problems and limitations noted above can be overcome by practicing the invention disclosed herein. Methods and compositions are provided that offer solutions to the aforementioned obstacles to effective use of viral vectors for treatment. Provided herein are methods and compositions for treating a subject with a viral vector comprising any one of the viral vector constructs provided herein in combination with synthetic nanocarriers comprising an immunosuppressant in a myriad of different dosing regimens, in particular with a pre-dose and/or post-dose of the synthetic nanocarriers comprising an immunosuppressant. The methods and related compositions provided can allow for improved use of viral vectors and can result in a reduction of undesired immune responses, such as IgM and/or IgG immune responses, and/or result in improved efficacy, such as through increased transgene expression.

The invention will now be described in more detail below.

### B. Definitions

“Administering” or “administration” or “administer” means giving or dispensing a material to a subject in a manner that is pharmacologically useful. The term is intended to include “causing to be administered”. “Causing to be administered” means causing, urging, encouraging, aiding, inducing or directing, directly or indirectly, another party to administer the material. When a time period between administrations are referred to, the time period is the time between the initiation of the administrations except as otherwise described.

As used herein, “coadministering” refers to administration at the same time or substantially at the same time where a clinician would consider any time between administrations virtually nil or negligible as to the impact on the desired therapeutic outcome. In some embodiments of anyone of the methods provided herein, the coadministration is simultaneous administration. “Simultaneous” means that the administrations begin within 5, 4, 3, 2, 1 or fewer minutes each other. In some embodiments, no more than 5, 4, 3, 2, 1 or fewer minutes pass between the end of the administration of one composition and the beginning of the administration of another composition. In other embodiments, no more than 5, 4, 3, 2, 1 or fewer minutes pass between the beginning of the administration of one composition and the beginning of the administration of another composition (e.g., such as when the two compositions are given in a different location and/or via a different mode). In some embodiments, simultaneous means the administrations are begun at the same time. In other embodiments, the compositions are admixed and given to a subject. The synthetic nanocarriers comprising an immunosuppressant may be coadministered with the viral vector repeatedly, for example 2, 3, 4, 5 or more times.

In some embodiments of any one of the methods provided, a coadministration of the viral vector and synthetic nanocarriers comprising an immunosuppressant is preceded by, and/or followed by, the administration of synthetic nanocarriers comprising an immunosuppressant without the viral vector (a pre-dose or post-dose, respectively, of the synthetic nanocarriers comprising immunosuppressant). In some embodiments of any one of the methods provided, the pre-dose of synthetic nanocarriers comprising an immunosuppressant is administered 1, 2 or 3 days before the coadministration of synthetic nanocarriers comprising an immunosuppressant and viral vector. In some embodiments of any one of the methods provided, the post-dose of synthetic nanocarriers comprising an immunosuppressant is administered 1, 2 or 3 days after the coadministration of synthetic nanocarriers comprising an immunosuppressant and viral vector. In some embodiments of any one of the methods provided, more than one pre-dose and/or post-dose is administered with each coadministration. In some embodiments of any one of the methods provided, when the co-administration is repeated, each repeated dose is preceded by 1 or 2 or more pre-doses. In some embodiments of any one of the methods provided, when the co-administration is repeated, each repeated dose is followed by 1 or 2 or more post-doses. In some embodiments of any one of the methods provided, when more than one post-dose is administered with each coadministration, the post-doses are administered biweekly with each coadministration.

“Admix” as used herein refers to the mixing of two or more components such that the two or more components are present together in a composition and administration of the composition provides the two or more components to a subject. Any one of the coadministrations of any one of the methods provided herein can be administered as an admixture.

“Amount effective” in the context of a composition for administration to a subject as provided herein refers to an amount of the composition that produces one or more desired results in the subject, for example, the reduction or elimination of an immune response, such as an IgM and/or IgG immune response, against a viral vector and/or efficacious or increased transgene expression. The amount effective can be for in vitro or in vivo purposes. For in vivo purposes, the amount can be one that a clinician would believe may have a clinical benefit for a subject that may experience undesired immune responses as a result of administration of a viral vector. In any one of the methods provided herein, the composition(s) administered may be in any one of the amounts effective as provided herein.

Amounts effective can involve reducing the level of an undesired immune response, although in some embodiments, it involves preventing an undesired immune response altogether. Amounts effective can also involve delaying the occurrence of an undesired immune response. An amount effective can also be an amount that results in a desired therapeutic endpoint or a desired therapeutic result. In some embodiments of any one of the compositions and methods provided, the amount effective is one in which a desired immune response, such as the reduction or elimination of an immune response against a viral vector, such as an IgM and/or IgG response, and/or the generation of efficacious or increased transgene expression, persists in the subject for at least 1 month. This reduction or elimination or efficacious or increased expression may be measured locally or systemically. The achievement of any of the foregoing can be monitored by routine methods.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation.

Amounts effective can refer to a dose of a component of a single material or it can refer to a dose of a component of a number of materials. For example, when referring to an amount effective of an immunosuppressant, the amount can refer to a single dose of a material that includes the immunosuppressant or a number of doses of the same or different materials that include the immunosuppressant. Thus, as used herein, in some embodiments of any one of the methods or compositions provided, the amount effective of an immunosuppressant may be the amount of immunosuppressant in a coadministration step as provided herein without other administrations of the immunosuppressant. In other embodiments of any one of the methods or compositions provided, however, the amount of immunosuppressant is the total amount of immunosuppressant for a set of administrations, such as the total amount of immunosuppressant of a coadministration as provided herein in combination with an amount of immunosuppressant of a pre-dose and/or post-dose as provided herein.

In some embodiments of any one of the methods or compositions provided, the amount of immunosuppressant is “split” amongst the set of administrations, and the total amount may be based on an amount determined to achieve a reduced immune response or efficacious or increased transgene expression of a viral vector according to another regimen, such as when coadministered with synthetic nanocarriers comprising an immunosuppressant but without the administration of a pre-dose or post-dose. This total amount of immunosuppressant can be administered according to a regimen as provided herein distributed amongst the amount of immunosuppressant given as a pre-dose and/or post-dose as well as the amount of immunosuppressant given as a coadministration step. Thus, in some embodiments of any one of the methods or compositions provided, the amount of immunosuppressant of the pre-dose and/or post-dose in combination with a coadministered dose is equal to this total.

In some embodiments of any one of the methods or compositions provided, the amount of immunosuppressant of a pre-dose or post-dose is no more than half of this total. In some embodiments of any one of the methods or compositions provided, the amount of immunosuppressant of a pre-dose or post-dose is half of this total. In some embodiments of any one of the methods or compositions provided, the amount of immunosuppressant of the pre-doses and/or post-doses may be the same as the amount of the immunosuppressant of the coadministering step.

“Assessing an immune response” refers to any measurement or determination of the level, presence or absence, reduction in, increase in, etc. of an immune response in vitro or in vivo. Such measurements or determinations may be performed on one or more samples obtained from a subject. Such assessing can be performed with any one of the methods provided herein or otherwise known in the art, including an ELISA-based assay. The assessing may be assessing the number or percentage of antibodies, such as IgM and/or IgG antibodies, such as those specific to a viral vector, such as in a sample from a subject. The assessing also may be assessing any effect related to the immune response, such as measuring the presence or absence of a cytokine, cell phenotype, etc. Any one of the methods provided herein may comprise or further comprise a step of assessing an immune response to a viral vector or antigen thereof. The assessing may be done directly or indirectly. The term is intended to include actions that cause, urge, encourage, aid, induce or direct another party to assess an immune response.

“Average”, as used herein, refers to the arithmetic mean unless otherwise noted.

“Couple” or “Coupled” (and the like) means to chemically associate one entity (for example a moiety) with another. In some embodiments of any one of the methods or compositions provided, the coupling is covalent, meaning that the attachment occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments of any one of the methods or compositions provided, encapsulation is a form of coupling.

“Dose” refers to a specific quantity of a pharmacologically and/or immunologically active material for administration to a subject for a given time. In general, doses of the synthetic nanocarriers comprising an immunosuppressant and/or viral vectors in the methods and compositions, which include kits, of the invention refer to the amount of immunosuppressant comprised in the synthetic nanocarriers and/or the amount of viral vectors unless otherwise provided. Alternatively, the dose can be administered based on the number of synthetic nanocarriers that provide the desired amount of an immunosuppressant, in instances when referring to a dose of synthetic nanocarriers that comprise an immunosuppressant. When dose is used in the context of a repeated dosing, dose refers to the amount of each of the repeated doses, which may be the same or different. A “pre-dose”, as used herein refers to a material or set of materials that is administered before an administration of another material or set of materials. A “post-dose”, as used herein, refers to a material or set of materials that is administered after an administration of another material or set of materials. In some embodiments of any one of the methods or compositions provided, the material(s) of a pre-dose or post-dose may be the same or different as the material(s) of the other administration. Preferably, as provided herein, the material of the pre-dose or post-dose comprises synthetic nanocarriers comprising an immunosuppressant but not comprising a viral vector.

“Encapsulate” means to enclose at least a portion of a substance within a synthetic nanocarrier. In some embodiments of any one of the methods or compositions provided, a substance is enclosed completely within a synthetic nanocarrier. In other embodiments of any one of the methods or compositions provided, most or all of a substance that is encapsulated is not exposed to the local environment external to the synthetic nanocarrier. In other embodiments of any one of the methods or compositions provided, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier.

“Expression control sequences” are any sequences that can affect expression and can include promoters, enhancers, and operators. Expression control sequences, or control elements, within vectors can facilitate proper nucleic acid transcription, translation, viral packaging, etc. Generally, control elements act in cis, but they may also work in trans. In one embodiment of any one of the methods or compositions provided, the expression control sequence is a promoter, such as a constitutive promoter or tissue-specific promoter. “Constitutive promoters,” also called ubiquitous or promiscuous promoters, are those that are thought of being generally active and not exclusive or preferential to certain cells. “Tissue-specific promoters” are those that are active in a particular cell type or tissue, such activity may be exclusive to the particular cell type or tissue. In any one of the nucleic acids or viral vectors provided herein the promoter may be any one of the promoters provided herein.

“Immune response against a viral vector” or the like refers to any undesired immune response against a viral vector, such as an antibody (e.g., IgM or IgG) or cellular response. In some embodiments, the undesired immune response is an antigen-specific immune response against the viral vector or an antigen thereof. In some embodiments, the immune response is specific to a viral antigen of the viral vector. In other embodiments, the immune response is specific to a protein or peptide encoded by a transgene of the viral vector. In some embodiments, the immune response is specific to a viral antigen of the viral vector and not to a protein or peptide that is encoded by a transgene of the viral vector.

In some embodiments, a reduced anti-viral vector response in a subject comprises a reduced anti-viral vector immune response measured using a biological sample obtained from the subject following administration as provided herein as compared to an anti-viral vector immune response measured using a biological sample obtained from another subject, such as a test subject, following administration to this other subject of the viral vector without administration as provided herein. In some embodiments, the anti-viral vector immune response is a reduced anti-viral vector immune response in a biological sample obtained from the subject following administration as provided herein upon a subsequent viral vector in vitro challenge performed on the subject's biological sample as compared to the anti-viral vector immune response detected upon viral vector in vitro challenge performed on a biological sample obtained from another subject, such as a test subject, following administration to the other subject of the viral vector without administration as provided herein. In other embodiments, an immune response can be assessed in another subject, such as in a sample from a test subject, where the results for the other subject, with or without scaling, would be expected to be indicative of what is occurring or has occurred in the subject at issue. In some embodiments, a reduced anti-viral vector response in a subject comprises a reduced anti-viral vector immune response measured using a biological sample obtained from the subject following administration as provided herein as compared to an anti-viral vector immune response measured using a biological sample obtained from the subject at a different point in time, such as at a time without administration as provided herein, for example, prior to an administration as provided herein.

“Immunosuppressant” means a compound that can cause a tolerogenic effect, preferably through its effects on APCs. A tolerogenic effect generally refers to the modulation by the APC or other immune cells systemically and/or locally, that reduces, inhibits or prevents an undesired immune response to an antigen in a durable fashion. In one embodiment of any one of the methods or compositions provided, the immunosuppressant is one that causes an APC to promote a regulatory phenotype in one or more immune effector cells. For example, the regulatory phenotype may be characterized by the inhibition of the production, induction, stimulation or recruitment of antigen-specific CD4+ T cells or B cells, the inhibition of the production of antigen-specific antibodies, the production, induction, stimulation or recruitment of Treg cells (e.g., CD4+CD25highFoxP3+ Treg cells), etc. This may be the result of the conversion of CD4+ T cells or B cells to a regulatory phenotype. This may also be the result of induction of FoxP3 in other immune cells, such as CD8+ T cells, macrophages and iNKT cells. In one embodiment of any one of the methods or compositions provided, the immunosuppressant is one that affects the response of the APC after it processes an antigen. In another embodiment of any one of the methods or compositions provided, the immunosuppressant is not one that interferes with the processing of the antigen. In a further embodiment of any one of the methods or compositions provided, the immunosuppressant is not an apoptotic-signaling molecule. In another embodiment of any one of the methods or compositions provided, the immunosuppressant is not a phospholipid.

Immunosuppressants include, but are not limited to, statins; mTOR inhibitors, such as rapamycin or a rapamycin analog (i.e., rapalog); TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, retinoic acid, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide. Other exemplary immunosuppressants include, but are not limited, small molecule drugs, natural products, antibodies (e.g., antibodies against CD20, CD3, CD4), biologics-based drugs, carbohydrate-based drugs, RNAi, antisense nucleic acids, aptamers, methotrexate, NSAIDs; fingolimod; natalizumab; alemtuzumab; anti-CD3; tacrolimus (FK506), abatacept, belatacept, etc. “Rapalog”, as used herein, refers to a molecule that is structurally related to (an analog) of rapamycin (sirolimus). Examples of rapalogs include, without limitation, temsirolimus (CCI-779), everolimus (RAD001), ridaforolimus (AP-23573), and zotarolimus (ABT-578). Additional examples of rapalogs may be found, for example, in WO Publication WO 1998/002441 and U.S. Pat. No. 8,455,510, the rapalogs of which are incorporated herein by reference in their entirety. Further immunosuppressants are known to those of skill in the art, and the invention is not limited in this respect. In embodiments of any one of the methods or compositions provided, the immunosuppressant may comprise any one of the agents provided herein, such as any one of the foregoing.

“Increasing transgene expression” refers to increasing the level of transgene expression of a viral vector in a subject, a transgene being delivered by the viral vector. In some embodiments, the level of the transgene expression may be determined by measuring transgene protein concentrations in various tissues or systems of interest in the subject. Alternatively, when the transgene expression product is a nucleic acid, the level of transgene expression may be measured by transgene nucleic acid products. Increasing transgene expression can be determined, for example, by measuring the amount of the transgene expression in a sample obtained from a subject and comparing it to a prior sample. The sample may be a tissue sample. In some embodiments, the transgene expression can be measured using flow cytometry. In other embodiments, increased transgene expression can be assessed in another subject, such as in a sample from a test subject, where the results for the other subject, with or without scaling, would be expected to be indicative of what is occurring or has occurred in the subject at issue. Any one of the methods provided herein may result in increased transgene expression.

“Load”, when an immunosuppressant is comprised in synthetic nanocarriers, such as when coupled thereto, is the amount of the immunosuppressant in the synthetic nanocarriers based on the total dry recipe weight of materials in an entire synthetic nanocarrier (weight/weight). Generally, such a load is calculated as an average across a population of synthetic nanocarriers. In one embodiment of any one of the methods or compositions provided the load on average across the synthetic nanocarriers is between 0.1% and 99%. In another embodiment of any one of the methods or compositions provided, the load is between 0.1% and 50%. In another embodiment of any one of the methods or compositions provided, the load is between 0.1% and 20%. In a further embodiment of any one of the methods or compositions provided, the load is between 0.1% and 10%. In still a further embodiment of any one of the methods or compositions provided, the load is between 1% and 10%. In still a further embodiment of any one of the methods or compositions provided, the load is between 7% and 20%. In yet another embodiment of any one of the methods or compositions provided, the load is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 40%, at least 50%, at least 60%, at least 70%, at least 80%, at least 90%, at least 95%, at least 96%, at least 97%, at least 98% or at least 99% on average across the population of synthetic nanocarriers. In yet a further embodiment of any one of the methods or compositions provided, the load is 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20% on average across the population of synthetic nanocarriers. In some embodiments of any one of the above embodiments, the load is no more than 25% on average across a population of synthetic nanocarriers. In embodiments of any one of the methods or compositions provided, the load is calculated as known in the art.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., effective diameter) may be obtained, in some embodiments, by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicies of the sample. The effective diameter, or mean of the distribution, is then reported. Determining the effective sizes of high aspect ratio, or non-spheroidal, synthetic nanocarriers may require augmentative techniques, such as electron microscopy, to obtain more accurate measurements. “Dimension” or “size” or “diameter” of synthetic nanocarriers means the mean of a particle size distribution, for example, obtained using dynamic light scattering.

“Pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Repeat dose” or “repeat dosing” or the like means at least one additional dose or dosing of a material or a set of materials that is administered to a subject subsequent to an earlier dose or dosing of the same material(s). While the material may be the same, the amount of the material in the repeated dose or dosing may be different.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like. As used herein, a subject may be in one need of any one of the methods or compositions provided herein. “Second subject” or “another subject” provided herein refers to another subject different from the subject to which the administrations are being provided. This subject can be any other subject, such as a test subject, which subject may be of the same or different species. Preferably, this second subject is one where a reduced immune response to a viral vector or efficacious or increased transgene expression of a viral vector has been achieved with a coadministration of the immunosuppressant comprised in the synthetic nanocarriers and the viral vector without having received a pre-dose or a post-dose of the immunosuppressant comprised in the synthetic nanocarriers. Thus, in some embodiments of any one of the methods provided, the second subject or another subject has only received the coadministration in order to achieve reduced immune response or increased transgene expression. The amount of the immunosuppressant of this coadministration can be used to determine the doses as provided herein for use according to any one of the described methods or in any one of the compositions provided herein. This amount can be distributed between the pre-doses and/or post-doses and coadministered doses to achieve a similar or greater effect.

In some embodiments of any one of the methods or compositions provided, when the second or other subject is of a different species the amount can be scaled as appropriate for the species of the subject to receive the administrations, which scaled amount can be used as the total as provided herein. For example, allometric scaling or other scaling methods can be used. Immune responses in second subjects or other subjects as well as transgene expression can be assessed using routine methods known to those of ordinary skill in the art or as otherwise provided herein. Any one of the methods provided herein may comprise or further comprise determining one or more of these amounts in a second or other subject as described herein.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, synthetic nanocarriers do not comprise chitosan. In other embodiments, synthetic nanocarriers are not lipid-based nanoparticles. In further embodiments, synthetic nanocarriers do not comprise a phospholipid.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid attached virus-like particles disclosed in published US Patent Application 20060251677 to Bachmann et al., (9) the virus-like particles disclosed in WO2010047839A1 or WO2009106999A2, (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), (11) apoptotic cells, apoptotic bodies or the synthetic or semisynthetic mimics disclosed in U.S. Publication 2002/0086049, or (12) those of Look et al., Nanogel-based delivery of mycophenolic acid ameliorates systemic lupus erythematosus in mice” J. Clinical Investigation 123(4):1741-1749 (2013).

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, synthetic nanocarriers may possess an aspect ratio greater than 1:1, 1:1.2, 1:1.5, 1:2, 1:3, 1:5, 1:7, or greater than 1:10.

“Transgene of the viral vector” or “transgene” or the like refers to nucleic acid material the viral vector is used to transport into a cell and, once in the cell, is expressed to produce a protein or nucleic acid molecule, respectively, such as for a therapeutic application as described herein. “Expressed” or “expression” or the like refers to the synthesis of a functional (i.e., physiologically active for the desired purpose) gene product after the transgene is transduced into a cell and processed by the transduced cell. Such a gene product is also referred to herein as a “transgene expression product”. The expressed products are, therefore, the resultant protein or nucleic acid, such as an antisense oligonucleotide or a therapeutic RNA, encoded by the transgene.

“Viral vector” means a viral-based delivery system that can or does deliver a payload, such as nucleic acid(s), to cells. Generally, the term refers to a viral vector construct with viral components, such as capsid and/or coat proteins, that can or does also comprise a payload (and has been so adapted). In some embodiments, the payload encodes a transgene. In some embodiments, a transgene is one that encodes a protein provided herein, such as a therapeutic protein, a DNA-binding protein or an endonuclease. In other embodiments, a transgene encodes guide RNA, an antisense nucleic acid, snRNA, an RNAi molecule (e.g., dsRNAs or ssRNAs), miRNA, or triplex-forming oligonucleotides (TFOs), etc. In other embodiments, the payload are nucleic acid(s) that themselves are the therapeutic(s) and expression of the delivered nucleic acid(s) is not required. For example, the nucleic acid(s) may be siRNA, such as synthetic siRNA.

In some embodiments, the payload may also encode other components such as inverted terminal repeats (ITRs), markers, etc. The payload may also include an expression control sequence. Expression control DNA sequences include promoters, enhancers, and operators, and are generally selected based on the expression systems in which the expression construct is to be utilized. In some embodiments, promoter and enhancer sequences are selected for the ability to increase gene expression, while operator sequences may be selected for the ability to regulate gene expression. The payload may also include sequences that facilitate, and preferably promote, homologous recombination in a host cell, in some embodiments.

Exemplary expression control sequences include promoter sequences, e.g., cytomegalovirus promoter; Rous sarcoma virus promoter; and simian virus 40 promoter; as well as any other types of promoters that are disclosed elsewhere herein or are otherwise known in the art. Generally, promoters are operatively linked upstream (i.e., 5′) of a sequence coding for a desired expression product. Payloads also may include a suitable polyadenylation sequence (e.g., the SV40 or human growth hormone gene polyadenylation sequence) operably linked downstream (i.e., 3′) of the coding sequence.

Generally, viral vectors are engineered to be capable of transducing one or more desired nucleic acids into a cell. In addition, it will be understood that for the therapeutic applications provided herein, it is preferred that the viral vectors be replication-defective. Viral vectors can be based on, without limitation, retroviruses (e.g., murine retrovirus, avian retrovirus, Moloney murine leukemia virus (MoMuLV), Harvey murine sarcoma virus (HaMuSV), murine mammary tumor virus (MuMTV), gibbon ape leukemia virus (GaLV) and Rous Sarcoma Virus (RSV)), lentiviruses, herpes viruses, adenoviruses, adeno-associated viruses, alphaviruses, etc. Other examples are provided elsewhere herein or are known in the art. The viral vectors may be based on natural variants, strains, or serotypes of viruses, such as any one of those provided herein. The viral vectors may also be based on viruses selected through molecular evolution (see, e.g., J. T. Koerber et al, Mol. Ther. 17(12):2088-2095 and U.S. Pat. No. 609,548). Viral vectors can be based on, without limitation, adeno-associated viruses (AAV), such as AAV8 or AAV2. Viral vectors can also be based on Anc80. Thus, an AAV vector or Anc80 vector provided herein is a viral vector based on an AAV or Anc80, respectively, and has viral components, such as a capsid and/or coat protein, therefrom that can package for delivery nucleic acid material. Other examples of AAV vectors include, but are not limited to, those based on AAV1, AAV2, AAV3, AAV4, AAV5, AAV6, AAV7, AAV8, AAV9, AAV10, AAV11, Rh10, Rh74, or AAV-2i8 or variants thereof. The viral vectors may also be engineered vectors, recombinant vectors, mutant vectors, or hybrid vectors. Methods of generating such vectors will be evident to one of ordinary skill in the art. In some embodiments, the viral vector is a “chimeric viral vector”. In such embodiments, this means that the viral vector is made up of viral components that are derived from more than one virus or viral vector. See, e.g., PCT Publications WO01/091802 and WO14/168953, and U.S. Pat. No. 6,468,771. Such a viral vector may be, for example, an AAV8/Anc80 or AAV2/Anc80 viral vector.

Additional viral vector elements may function in cis or in trans. In some embodiments, the viral vector includes a vector genome that also includes one or more inverted terminal repeat (ITR) sequence(s) that flank that 5′ or 3′ terminus of the target (donor) sequence, an expression control element that promotes transcription (e.g., promoter or enhancer), an intron sequence, a stuffer/filler polynucleotide sequence (generally, an inert sequence), and/or a poly(A) sequence located at the 3′ end of the target (donor) sequence.

### C. Compositions for Use in the Inventive Methods

Importantly, the methods and compositions provided herein provide improved effects with administration of viral vectors. Thus, the methods and compositions provided herein are useful for the treatment of subjects with viral vectors. Such viral vectors can be used to deliver nucleic acids for a variety of purposes, including for gene therapy, etc. As mentioned above, immune responses against a viral vector can adversely impact its efficacy and can also interfere with its readministration. Importantly, the methods and compositions provided herein have been found to overcome the aforementioned obstacles by achieving improved expression of transgenes and/or reducing immune responses to viral vectors. The inventors have surprisingly discovered that dosing regimens that include a pre-dose and/or post-dose of synthetic nanocarriers comprising an immunosuppressant in combination with a coadministration of the synthetic nanocarriers and viral vector can achieve improved immune response reduction and/or transgene expression. In addition, it was also surprisingly found that the amount of immunosuppressant, when comprised in synthetic nanocarriers, of a coadministration step could be reduced with a pre-dose or post-dose as compared to the coadministration step alone. Thus, an amount of immunosuppressant, when comprised in synthetic nanocarriers, can be “split” amongst a pre-dose and/or post-dose and coadministered dose in any one of the treatment regimens provided herein.

Also as mentioned above, it has been discovered that viral vector administration can result in IgM immune responses shortly after viral vector administration. It has also been discovered that synthetic nanocarriers comprising an immunosuppressant and administered at times relative to the viral vector can induce elevated transgene expression in an IgM-dependent manner.

### Transgenes

The payload of a viral vector may be a transgene. For example, the transgene may encode a desired expression product, such as a polypeptide, protein, protein mixture, DNA, cDNA, functional RNA molecule (e.g., RNAi, miRNA), mRNA, RNA replicon, or other product of interest.

For example, the expression product of the transgene may be a protein or portion thereof beneficial to a subject, such as one with a disease or disorder. The protein may be an extracellular, intracellular or membrane-bound protein. Transgenes, for example, may encode enzymes, blood derivatives, hormones, lymphokines, such as the interleukins and interferons, coagulants, growth factors, neurotransmitters, tumor suppressors, apolipoproteins, antigens, and antibodies. The subject may have or be suspected of having a disease or disorder whereby the subject's endogenous version of the protein is defective or produced in limited amounts or not at all. In other embodiments of any one of the methods or compositions provided, the expression product of the transgene may be a gene or portion thereof beneficial to a subject.

Examples of therapeutic proteins include, but are not limited to, infusible or injectable therapeutic proteins, enzymes, enzyme cofactors, hormones, blood or blood coagulation factors, cytokines and interferons, growth factors, adipokines, etc.

Examples of infusible or injectable therapeutic proteins include, for example, Tocilizumab (Roche/Actemra®), alpha-1 antitryp sin (Kamada/AAT), Hematide® (Affymax and Takeda, synthetic peptide), albinterferon alfa-2b (Novartis/Zalbin™), Rhucin® (Pharming Group, C1 inhibitor replacement therapy), tesamorelin (Theratechnologies/Egrifta, synthetic growth hormone-releasing factor), ocrelizumab (Genentech, Roche and Biogen), belimumab (GlaxoSmithKline/Benlysta®), pegloticase (Savient Pharmaceuticals/Krystexxa™), taliglucerase alfa (Protalix/Uplyso), agalsidase alfa (Shire/Replagal®), and velaglucerase alfa (Shire).

Examples of enzymes include lysozyme, oxidoreductases, transferases, hydrolases, lyases, isomerases, asparaginases, uricases, glycosidases, proteases, nucleases, collagenases, hyaluronidases, heparinases, heparanases, kinases, phosphatases, lysins and ligases. Other examples of enzymes include those that used for enzyme replacement therapy including, but not limited to, imiglucerase (e.g., CEREZYME™), a-galactosidase A (a-gal A) (e.g., agalsidase beta, FABRYZYME™), acid a-glucosidase (GAA) (e.g., alglucosidase alfa, LUMIZYME™, MYOZYME™), and arylsulfatase B (e.g., laronidase, ALDURAZYME™, idursulfase, ELAPRASE™, arylsulfatase B, NAGLAZYME™).

Examples of hormones include Melatonin (N-acetyl-5-methoxytryptamine), Serotonin, Thyroxine (or tetraiodothyronine) (a thyroid hormone), Triiodothyronine (a thyroid hormone), Epinephrine (or adrenaline), Norepinephrine (or noradrenaline), Dopamine (or prolactin inhibiting hormone), Antimullerian hormone (or mullerian inhibiting factor or hormone), Adiponectin, Adrenocorticotropic hormone (or corticotropin), Angiotensinogen and angiotensin, Antidiuretic hormone (or vasopres sin, arginine vasopres sin), Atrial-natriuretic peptide (or atriopeptin), Calcitonin, Cholecystokinin, Corticotropin-releasing hormone, Erythropoietin, Follicle-stimulating hormone, Gastrin, Ghrelin, Glucagon, Glucagon-like peptide (GLP-1), GIP, Gonadotropin-releasing hormone, Growth hormone-releasing hormone, Human chorionic gonadotropin, Human placental lactogen, Growth hormone, Inhibin, Insulin, Insulin-like growth factor (or somatomedin), Leptin, Luteinizing hormone, Melanocyte stimulating hormone, Orexin, Oxytocin, Parathyroid hormone, Prolactin, Relaxin, Secretin, Somatostatin, Thrombopoietin, Thyroid-stimulating hormone (or thyrotropin), Thyrotropin-releasing hormone, Cortisol, Aldosterone, Testosterone, Dehydroepiandrosterone, Androstenedione, Dihydrotestosterone, Estradiol, Estrone, Estriol, Progesterone, Calcitriol (1,25-dihydroxyvitamin D3), Calcidiol (25-hydroxyvitamin D3), Prostaglandins, Leukotrienes, Prostacyclin, Thromboxane, Prolactin releasing hormone, Lipotropin, Brain natriuretic peptide, Neuropeptide Y, Histamine, Endothelin, Pancreatic polypeptide, Renin, and Enkephalin.

Examples of blood or blood coagulation factors include Factor I (fibrinogen), Factor II (prothrombin), tissue factor, Factor V (proaccelerin, labile factor), Factor VII (stable factor, proconvertin), Factor VIII (antihemophilic globulin), Factor IX (Christmas factor or plasma thromboplastin component), Factor X (Stuart-Prower factor), Factor Xa, Factor XI, Factor XII (Hageman factor), Factor XIII (fibrin-stabilizing factor), von Willebrand factor, von Heldebrant Factor, prekallikrein (Fletcher factor), high-molecular weight kininogen (HMWK) (Fitzgerald factor), fibronectin, fibrin, thrombin, antithrombin, such as antithrombin III, heparin cofactor II, protein C, protein S, protein Z, protein Z-related protease inhibitot (ZPI), plasminogen, alpha 2-antiplasmin, tissue plasminogen activator (tPA), urokinase, plasminogen activator inhibitor-1 (PAI1), plasminogen activator inhibitor-2 (PAI2), cancer procoagulant, and epoetin alfa (Epogen, Procrit).

Examples of cytokines include lymphokines, interleukins, and chemokines, type 1 cytokines, such as IFN-γ, TGF-β, and type 2 cytokines, such as IL-4, IL-10, and IL-13.

Examples of growth factors include Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Transforming growth factor beta(TGF-β), Tumour necrosis factor-alpha(TNF-α), Vascular endothelial growth factor (VEGF), Wnt Signaling Pathway, placental growth factor (P1GF), [(Foetal Bovine Somatotrophin)] (FBS), IL-1, IL-2, IL-3, IL-4, IL-5, IL-6, and IL-7.

Examples of adipokines, include leptin and adiponectin.

Additional examples of therapeutic proteins include, but are not limited to, receptors, signaling proteins, cytoskeletal proteins, scaffold proteins, transcription factors, structural proteins, membrane proteins, cytosolic proteins, binding proteins, nuclear proteins, secreted proteins, Golgi proteins, endoplasmic reticulum proteins, mitochondrial proteins, and vesicular proteins, etc.

In one embodiment of any one of the methods or compositions provided, the expression product may be used to disrupt, correct/repair, or replace a target gene, or part of a target gene. For example, the Clustered Regularly Interspaced Short Palindromic Repeat/Cas (CRISPR/Cas) system can be used for precise genome editing. In the system, single CRISPR-associated nucleases (Cas nucleases) may be programmed by a guide RNA (short RNA) to recognize a specific DNA target, which comprises DNA loci containing short repetitions of a base sequence. Each CRISPR loci is flanked by short segment of spacer DNA, which are derived from viral genomic material. In the type II CRISPR system, the most common system, spacer DNA hybridizes with trans-activating RNA (tracRNA), where it is processed into CRISPR-RNA (crRNA) and then associates with Cas nucleases, forming complexes which initiate RNAse III processing and resulting in the degradation of foreign DNA. The target sequence preferably contains a protospacer adjacent motif (PAM) sequence on its 3′ end in order to be recognized. The system can be modified in a number of ways, for example synthetic guide RNAs may be fused to a CRISPR vector, and a variety of different guide RNA structures and elements are possible (including hairpin and scaffold sequences).

In some embodiments of any one of the methods or compositions provided, the transgene sequence may encode any one or more components of a CRISPR/Cas system, such as a reporter sequence, which produces a detectable signal when expressed. Examples of such reporters sequences include, but are not limited to, β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, and the influenza hemagglutinin protein. Other reporters are known to those of ordinary skill in the art.

In another example of any one of the methods or compositions provided, the transgene may encode an RNA product, such as tRNA, dsRNA, ribosomal RNA, catalytic RNAs, siRNA, RNAi, miRNA, small hairpin RNA (shRNA), trans-splicing RNA, and antisense RNAs. For example, specific RNA sequences can be generated to inhibit or extinguish the expression of a targeted nucleic acid sequence in the subject. Suitable target sequences include, for example, oncologic targets and viral diseases.

In some embodiments of any one of the methods or compositions provided, the transgene sequence may encode a reporter sequence, which produces a detectable signal when expressed, or the transgene sequence may encode a protein or functional RNA that can be used to create an animal model of disease. In another example of any one of the methods or compositions provided, the transgene encodes a protein or functional RNA that is intended to be used for research purposes, e.g., to create a somatic transgenic animal model harboring the transgene, e.g., to study the function of the transgene product. In other embodiments of any one of the methods or compositions provided, the intent of such expression products is for treatment. Other uses of transgenes will be apparent to one of ordinary skill in the art.

The sequence of a transgene may also include an expression control sequence. Expression control sequences include promoters, enhancers, and operators, and are generally selected based on the expression systems in which the expression construct is to be utilized. In some embodiments of any one of the methods or compositions provided, promoter and enhancer sequences are selected for the ability to increase gene expression, while operator sequences may be selected for the ability to regulate gene expression. Typically, promoter sequences are located upstream (i.e., 5′) of the nucleic acid sequence encoding the desired expression product, and are operatively linked to an adjacent sequence, thereby increasing the amount of desired product expressed as compared to an amount expressed without the promoter. Enhancer sequences, generally located upstream of promoter sequences, can further increase expression of the desired product. In some embodiments of any one of the methods or compositions provided, the enhancer sequence(s) may be located downstream of the promoter and/or within the transgene. The transgene may also include sequences that facilitate, and preferably promote, homologous recombination in a host cell and/or packaging. The transgene may also include sequences that are necessary for replication in a host cell.

Exemplary expression control sequences include liver-specific promoter sequences and constitutive promoter sequences, such as any one that may be provided herein. Other tissue-specific promoters include eye, retina, central nervous system, spinal cord, among others. Examples of ubiquitous or promiscuous promoters and enhancers include, but are not limited to the cytomegalovirus (CMV) immediate early promoter/enhancer sequences, the Rous sarcoma virus (RSV) promoter/enhancer sequences and the other viral promoters/enhancers active in various mammalian cell types, or synthetic elements that are not present in nature (see, e.g., Boshart et al, Cell, 41:521-530 (1985)), the SV40 promoter, the dihydrofolate reductase (DHFR) promoter, the cytoplasmic β-actin promoter and the phosphoglycerol kinase (PGK) promoter.

Operators, or regulatable elements, are responsive to a signal or stimuli, which can increase or decrease the expression of the operably linked nucleic acid. Inducible elements are those that increase the expression of the operably linked nucleic acid in response to a signal or stimuli, for example, hormone inducible promoters. Repressible elements are those that decrease the expression of the operably linked nucleic acid in response to a signal or stimuli. Typically, repressible and inducible elements are proportionally responsive to the amount of signal or stimuli present. The transgene may include such sequences in any one of the methods or compositions provided.

The transgene also may include a suitable polyadenylation sequence operably linked downstream (i.e., 3′) of the coding sequence.

Methods of delivering transgenes, for example, for gene therapy, are known in the art (see, e.g., Smith. Int. J. Med. Sci. 1(2): 76-91 (2004); Phillips. Methods in Enzymology: Gene Therapy Methods. Vol. 346. Academic Press (2002)). Any of the transgenes described herein may be incorporated into any of the viral vectors described herein using methods of known in the art, see, for example, U.S. Pat. No. 7,629,153.

### Viral Vectors

Viruses have evolved specialized mechanisms to transport their genomes inside the cells that they infect; viral vectors based on such viruses can be tailored to transduce cells to specific applications. Examples of viral vectors that may be used as provided herein are known in the art or described herein. Suitable viral vectors include, for instance, retroviral vectors, lentiviral vectors, herpes simplex virus (HSV)-based vectors, adenovirus-based vectors, adeno-associated virus (AAV)-based vectors, and AAV-adenoviral chimeric vectors.

The viral vectors provided herein may be based on a retrovirus. Retrovirus is a single-stranded positive sense RNA virus. A retroviral vector can be manipulated to render the virus replication-incompetent. As such, retroviral vectors are thought to be particularly useful for stable gene transfer in vivo. Examples of retroviral vectors can be found, for example, in U.S. Publication Nos. 20120009161, 20090118212, and 20090017543, the viral vectors and methods of their making being incorporated by reference herein in their entirety.

Lentiviral vectors are examples of retroviral vectors that can be used for the production of a viral vector as provided herein. Examples of lentiviruses include HIV (humans), simian immunodeficiency virus (SIV), feline immunodeficiency virus (FIV), equine infectious anemia virus (EIAV) and visna virus (ovine lentivirus). Examples of lentiviral vectors can be found, for example, in U.S. Publication Nos. 20150224209, 20150203870, 20140335607, 20140248306, 20090148936, and 20080254008, the viral vectors and methods of their making being incorporated by reference herein in their entirety.

Herpes simplex virus (HSV)-based viral vectors are also suitable for use as provided herein. Many replication-deficient HSV vectors contain a deletion to remove one or more intermediate-early genes to prevent replication. For a description of HSV-based vectors, see, for example, U.S. Pat. Nos. 5,837,532, 5,846,782, 5,849,572, and 5,804,413, and International Patent Applications WO 91/02788, WO 96/04394, WO 98/15637, and WO 99/06583, the description of which viral vectors and methods of their making being incorporated by reference in its entirety.

Viral vectors can be based on adenoviruses. The adenovirus on which a viral vector may be based may be from any origin, any subgroup, any subtype, mixture of subtypes, or any serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087. Any adenovirus, even a chimeric adenovirus, can be used as the source of the viral genome for an adenoviral vector. For example, a human adenovirus can be used as the source of the viral genome for a replication-deficient adenoviral vector. Further examples of adenoviral vectors can be found in U.S. Publication Nos. 20150093831, 20140248305, 20120283318, 20100008889, 20090175897 and 20090088398, the description of which viral vectors and methods of their making being incorporated by reference in its entirety.

The viral vectors provided herein can also be based on adeno-associated viruses (AAVs). AAV vectors have been of particular interest for use in therapeutic applications such as those described herein. For a description of AAV-based vectors, see, for example, U.S. Pat. Nos. 8,679,837, 8,637,255, 8,409,842, 7,803,622, and 7,790,449, and U.S. Publication Nos. 20150065562, 20140155469, 20140037585, 20130096182, 20120100606, and 20070036757. The AAV vectors may be recombinant AAV vectors. The AAV vectors may also be self-complementary (sc) AAV vectors, which are described, for example, in U.S. Patent Publications 2007/01110724 and 2004/0029106, and U.S. Pat. Nos. 7,465,583 and 7,186,699, the viral vectors of which and methods or their making being incorporated herein by reference in their entirety.

The adeno-associated virus on which a viral vector may be based may be of any serotype or a mixture of serotypes. AAV serotypes include AAV1, AAV2, AAV3, AAV4, AAVS, AAV6, AAV7, AAV8, AAV9, AAV10, and AAV11. For example, when the viral vector is based on a mixture of serotypes, the viral vector may contain the capsid signal sequences taken from one AAV serotype (for example selected from any one of AAV serotypes 1, 2, 3, 4, 5, 6, 7, 8, 9, 10, and 11) and packaging sequences from a different serotype (for example selected from any one of AAV serotypes 1, 2, 3, 4, 5, 6, 7, 8, 9, 10, and 11). In some embodiments of any one of the methods or compositions provided herein, therefore, the AAV vector is an AAV 2/8-based vector. In other embodiments of any one of the methods or compositions provided herein, the AAV vector is an AAV 2/5-based vector.

In some embodiments of any one of the methods or compositions provided, the virus on which a viral vector is based may be synthetic, such as Anc80.

In some embodiments of any one of the methods or compositions provided, the viral vector is an AAV/Anc80 vectors, such as an AAV8/Anc80 vector or an AAV2/Anc80 vector.

Other viruses on which the vector can be based include AAV1, AAV3, AAV4, AAVS, AAV6, AAV7, AAV9, AAV10, AAV11, rh10, rh74 or AAV-2i8, and variants thereof.

The viral vectors provided herein may also be based on an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Examples of alphaviral vectors can be found in U.S. Publication Nos. 20150050243, 20090305344, and 20060177819; the vectors and methods of their making are incorporated herein by reference in their entirety.

Any one of the viral vectors provided herein may be for use in any one of the methods provided herein.

### Immunosuppressants

Immunosuppressants include, but are not limited to, statins; mTOR inhibitors, such as rapamycin or a rapamycin analog; TGF-β signaling agents; TGF-β receptor agonists; histone deacetylase (HDAC) inhibitors; corticosteroids; inhibitors of mitochondrial function, such as rotenone; P38 inhibitors; NF-κβ inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cyclosporine A, siRNAs targeting cytokines or cytokine receptors and the like.

Examples of statins include atorvastatin (LIPITOR®, TORVAST®), cerivastatin, fluvastatin (LESCOL®, LESCOL® XL), lovastatin (MEVACOR®, ALTOCOR®, ALTOPREV®), mevastatin (COMPACTIN®), pitavastatin (LIVALO®, PIAVA®), rosuvastatin (PRAVACHOL®, SELEKTINE®, LIPOSTAT®), rosuvastatin (CRESTOR®), and simvastatin (ZOCOR®, LIPEX®.

Examples of mTOR inhibitors include rapamycin and analogs thereof (e.g., CCL-779, RAD001, AP23573, C20-methallylrapamycin (C20-Marap), C16-(S)-butylsulfonamidorapamycin (C16-BSrap), C16-(S)-3-methylindolerapamycin (C16-iRap) (Bayle et al. Chemistry & Biology 2006, 13:99-107)), AZD8055, BEZ235 (NVP-BEZ235), chrysophanic acid (chrysophanol), deforolimus (MK-8669), everolimus (RAD0001), KU-0063794, PI-103, PP242, temsirolimus, and WYE-354 (available from Selleck, Houston, Tex., USA).

Examples of TGF-β signaling agents include TGF-β ligands (e.g., activin A, GDF1, GDF11, bone morphogenic proteins, nodal, TGF-βs) and their receptors (e.g., ACVR1B, ACVR1C, ACVR2A, ACVR2B, BMPR2, BMPR1A, BMPR1B, TGFβRI, TGFβRII), R-SMADS/co-SMADS (e.g., SMAD1, SMAD2, SMAD3, SMAD4, SMAD5, SMAD8), and ligand inhibitors (e.g., follistatin, noggin, chordin, DAN, lefty, LTBP1, THBS1, Decorin).

Examples of inhibitors of mitochondrial function include atractyloside (dipotassium salt), bongkrekic acid (triammonium salt), carbonyl cyanide m-chlorophenylhydrazone, carboxyatractyloside (e.g., from Atractylis gummifera), CGP-37157, (−)-Deguelin (e.g., from Mundulea sericea), F16, hexokinase II VDAC binding domain peptide, oligomycin, rotenone, Ru360, SFK1, and valinomycin (e.g., from Streptomyces fulvissimus) (EMD4Biosciences, USA).

Examples of P38 inhibitors include SB-203580 (4-(4-Fluorophenyl)-2-(4-methylsulfinylphenyl)-5-(4-pyridyl)1H-imidazole), SB-239063 (trans-1-(4hydroxycyclohexyl)-4-(fluorophenyl)-5-(2-methoxy-pyrimidin-4-yl) imidazole), SB-220025 (5-(2amino-4-pyrimidinyl)-4-(4-fluorophenyl)-1-(4-piperidinyl)imidazole)), and ARRY-797.

Examples of NF (e.g., NK-κβ) inhibitors include IFRD1, 2-(1,8-naphthyridin-2-yl)-Phenol, 5-aminosalicylic acid, BAY 11-7082, BAY 11-7085, CAPE (Caffeic Acid Phenethylester), diethylmaleate, IKK-2 Inhibitor IV, IMD 0354, lactacystin, MG-132 [Z-Leu-Leu-Leu-CHO], NFκB Activation Inhibitor III, NF-κB Activation Inhibitor II, JSH-23, parthenolide, Phenylarsine Oxide (PAO), PPM-18, pyrrolidinedithiocarbamic acid ammonium salt, QNZ, RO 106-9920, rocaglamide, rocaglamide AL, rocaglamide C, rocaglamide I, rocaglamide J, rocaglaol, (R)-MG-132, sodium salicylate, triptolide (PG490), and wedelolactone.

Examples of adenosine receptor agonists include CGS-21680 and ATL-146e.

Examples of prostaglandin E2 agonists include E-Prostanoid 2 and E-Prostanoid 4.

Examples of phosphodiesterase inhibitors (non-selective and selective inhibitors) include caffeine, aminophylline, IBMX (3-isobutyl-1-methylxanthine), paraxanthine, pentoxifylline, theobromine, theophylline, methylated xanthines, vinpocetine, EHNA (erythro-9-(2-hydroxy-3-nonyl)adenine), anagrelide, enoximone (PERFAN™), milrinone, levosimendon, mesembrine, ibudilast, piclamilast, luteolin, drotaverine, roflumilast (DAXAS™, DALIRESP™), sildenafil (REVATION®, VIAGRA®), tadalafil (ADCIRCA®, CIALIS®), vardenafil (LEVITRA®, STAXYN®), udenafil, avanafil, icariin, 4-methylpiperazine, and pyrazolo pyrimidin-7-1.

Examples of proteasome inhibitors include bortezomib, disulfiram, epigallocatechin-3-gallate, and salinosporamide A.

Examples of kinase inhibitors include bevacizumab, BIBW 2992, cetuximab (ERBITUX®), imatinib (GLEEVEC®), trastuzumab (HERCEPTIN®), gefitinib (IRESSA®), ranibizumab (LUCENTIS®), pegaptanib, sorafenib, dasatinib, sunitinib, erlotinib, nilotinib, lapatinib, panitumumab, vandetanib, E7080, pazopanib, and mubritinib.

Examples of glucocorticoids include hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (DOCA), and aldosterone.

Examples of retinoids include retinol, retinal, tretinoin (retinoic acid, RETIN-A®), isotretinoin (ACCUTANE®, AMNESTEEM®, CLARAVIS®, SOTRET®), alitretinoin (PANRETIN®), etretinate (TEGISON™) and its metabolite acitretin (SORIATAN®), tazarotene (TAZORAC®, AVAGE®, ZORAC®), bexarotene (TARGRETIN®), and adapalene (DIFFERIN®).

Examples of cytokine inhibitors include IL1ra, IL1 receptor antagonist, IGFBP, TNF-BF, uromodulin, Alpha-2-Macroglobulin, Cyclosporin A, Pentamidine, and Pentoxifylline (PENTOPAK®, PENTOXIL®, TRENTAL®).

Examples of peroxisome proliferator-activated receptor antagonists include GW9662, PPARγ antagonist III, G335, and T0070907 (EMD4Biosciences, USA).

Examples of peroxisome proliferator-activated receptor agonists include pioglitazone, ciglitazone, clofibrate, GW1929, GW7647, L-165,041, LY 171883, PPARγ activator, Fmoc-Leu, troglitazone, and WY-14643 (EMD4Biosciences, USA).

Examples of histone deacetylase inhibitors include hydroxamic acids (or hydroxamates) such as trichostatin A, cyclic tetrapeptides (such as trapoxin B) and depsipeptides, benzamides, electrophilic ketones, aliphatic acid compounds such as phenylbutyrate and valproic acid, hydroxamic acids such as vorinostat (SAHA), belinostat (PXD101), LAQ824, and panobinostat (LBH589), benzamides such as entinostat (MS-275), CI994, and mocetinostat (MGCD0103), nicotinamide, derivatives of NAD, dihydrocoumarin, naphthopyranone, and 2-hydroxynaphaldehydes.

Examples of calcineurin inhibitors include cyclosporine, pimecrolimus, voclosporin, and tacrolimus.

Examples of phosphatase inhibitors include BN82002 hydrochloride, CP-91149, calyculin A, cantharidic acid, cantharidin, cypermethrin, ethyl-3,4-dephostatin, fostriecin sodium salt, MAZ51, methyl-3,4-dephostatin, NSC 95397, norcantharidin, okadaic acid ammonium salt from prorocentrum concavum, okadaic acid, okadaic acid potassium salt, okadaic acid sodium salt, phenylarsine oxide, various phosphatase inhibitor cocktails, protein phosphatase 1C, protein phosphatase 2A inhibitor protein, protein phosphatase 2A1, protein phosphatase 2A2, and sodium orthovanadate.

### Synthetic Nanocarriers

The methods provided herein include administrations of synthetic nanocarriers comprising an immunosuppressant. Generally, the immunosuppressant is an element that is in addition to the material that makes up the structure of the synthetic nanocarrier. For example, in one embodiment of any one of the methods or compositions provided, where the synthetic nanocarrier is made up of one or more polymers, the immunosuppressant is a compound that is in addition and, in some embodiments of any one of the methods or compositions provided, attached to the one or more polymers. In embodiments where the material of the synthetic nanocarrier also results in a tolerogenic effect, the immunosuppressant is an element present in addition to the material of the synthetic nanocarrier that results in a tolerogenic effect.

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size or shape so that each synthetic nanocarrier has similar properties. For example, at least 80%, at least 90%, or at least 95% of the synthetic nanocarriers of any one of the compositions or methods provided, based on the total number of synthetic nanocarriers, may have a minimum dimension or maximum dimension that falls within 5%, 10%, or 20% of the average diameter or average dimension of the synthetic nanocarriers.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, synthetic nanocarriers can comprise one or more polymers. In some embodiments, the synthetic nanocarriers comprise one or more polymers that is a non-methoxy-terminated, pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated, pluronic polymers. In some embodiments, all of the polymers that make up the synthetic nanocarriers are non-methoxy-terminated, pluronic polymers. In some embodiments, the synthetic nanocarriers comprise one or more polymers that is a non-methoxy-terminated polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated polymers. In some embodiments, all of the polymers that make up the synthetic nanocarriers are non-methoxy-terminated polymers. In some embodiments, the synthetic nanocarriers comprise one or more polymers that do not comprise pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers do not comprise pluronic polymer. In some embodiments, all of the polymers that make up the synthetic nanocarriers do not comprise pluronic polymer. In some embodiments, such a polymer can be surrounded by a coating layer (e.g., liposome, lipid monolayer, micelle, etc.). In some embodiments, elements of the synthetic nanocarriers can be attached to the polymer.

Immunosuppressants can be coupled to the synthetic nanocarriers by any of a number of methods. Generally, the attaching can be a result of bonding between the immunosuppressants and the synthetic nanocarriers. This bonding can result in the immunosuppressants being attached to the surface of the synthetic nanocarriers and/or contained (encapsulated) within the synthetic nanocarriers. In some embodiments, however, the immunosuppressants are encapsulated by the synthetic nanocarriers as a result of the structure of the synthetic nanocarriers rather than bonding to the synthetic nanocarriers. In preferable embodiments, the synthetic nanocarrier comprises a polymer as provided herein, and the immunosuppressants are attached to the polymer.

When attaching occurs as a result of bonding between the immunosuppressants and synthetic nanocarriers, the attaching may occur via a coupling moiety. A coupling moiety can be any moiety through which an immunosuppressant is bonded to a synthetic nanocarrier. Such moieties include covalent bonds, such as an amide bond or ester bond, as well as separate molecules that bond (covalently or non-covalently) the immunosuppressant to the synthetic nanocarrier. Such molecules include linkers or polymers or a unit thereof. For example, the coupling moiety can comprise a charged polymer to which an immunosuppressant electrostatically binds. As another example, the coupling moiety can comprise a polymer or unit thereof to which it is covalently bonded.

In preferred embodiments, the synthetic nanocarriers comprise a polymer as provided herein. These synthetic nanocarriers can be completely polymeric or they can be a mix of polymers and other materials.

In some embodiments, the polymers of a synthetic nanocarrier associate to form a polymeric matrix. In some of these embodiments, a component, such as an immunosuppressant, can be covalently associated with one or more polymers of the polymeric matrix. In some embodiments, covalent association is mediated by a linker. In some embodiments, a component can be noncovalently associated with one or more polymers of the polymeric matrix. For example, in some embodiments, a component can be encapsulated within, surrounded by, and/or dispersed throughout a polymeric matrix. Alternatively or additionally, a component can be associated with one or more polymers of a polymeric matrix by hydrophobic interactions, charge interactions, van der Waals forces, etc. A wide variety of polymers and methods for forming polymeric matrices therefrom are known conventionally.

Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments, the polymer comprises a polyester, polycarbonate, polyamide, or polyether, or unit thereof. In other embodiments, the polymer comprises poly(ethylene glycol) (PEG), polypropylene glycol, poly(lactic acid), poly(glycolic acid), poly(lactic-co-glycolic acid), or a polycaprolactone, or unit thereof. In some embodiments, it is preferred that the polymer is biodegradable. Therefore, in these embodiments, it is preferred that if the polymer comprises a polyether, such as poly(ethylene glycol) or polypropylene glycol or unit thereof, the polymer comprises a block-co-polymer of a polyether and a biodegradable polymer such that the polymer is biodegradable. In other embodiments, the polymer does not solely comprise a polyether or unit thereof, such as poly(ethylene glycol) or polypropylene glycol or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids. Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids. In embodiments, the synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

Compositions according to the invention can comprise pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, compositions are suspended in sterile saline solution for injection together with a preservative.

### D. Methods of Using and Making the Compositions

Viral vectors can be made with methods known to those of ordinary skill in the art or as otherwise described herein. For example, viral vectors can be constructed and/or purified using the methods set forth, for example, in U.S. Pat. No. 4,797,368 and Laughlin et al., Gene, 23, 65-73 (1983).

As an example, replication-deficient adenoviral vectors can be produced in complementing cell lines that provide gene functions not present in the replication-deficient adenoviral vectors, but required for viral propagation, at appropriate levels in order to generate high titers of viral vector stock. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons). Construction of complementing cell lines involve standard molecular biology and cell culture techniques, such as those described by Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Complementing cell lines for producing adenoviral vectors include, but are not limited to, HEK 293 cells (described in, e.g., Graham et al., J. Gen. Virol., 36, 59-72 (1977)), PER.C6 cells (described in, e.g., International Patent Application WO 97/00326, and U.S. Pat. Nos. 5,994,128 and 6,033,908), and 293-ORF6 cells (described in, e.g., International Patent Application WO 95/34671 and Brough et al., J. Virol., 71, 9206-9213 (1997)). In some instances, the complementing cell will not complement for all required adenoviral gene functions. Helper viruses can be employed to provide the gene functions in trans that are not encoded by the cellular or adenoviral genomes to enable replication of the adenoviral vector. Adenoviral vectors can be constructed, propagated, and/or purified using the materials and methods set forth, for example, in U.S. Pat. Nos. 5,965,358, 5,994,128, 6,033,908, 6,168,941, 6,329,200, 6,383,795, 6,440,728, 6,447,995, and 6,475,757, U.S. Patent Application Publication No. 2002/0034735 A1, and International Patent Applications WO 98/53087, WO 98/56937, WO 99/15686, WO 99/54441, WO 00/12765, WO 01/77304, and WO 02/29388, as well as the other references identified herein. Non-group C adenoviral vectors, including adenoviral serotype 35 vectors, can be produced using the methods set forth in, for example, U.S. Pat. Nos. 5,837,511 and 5,849,561, and International Patent Applications WO 97/12986 and WO 98/53087.

Viral vectors, such as AAV vectors, may be produced using recombinant methods. For example, the methods can involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, the viral vector may comprise inverted terminal repeats (ITR) of AAV serotypes selected from the group consisting of: AAV1, AAV2, AAVS, AAV6, AAV6.2, AAV7, AAV8, AAV9, AAV10, AAV11 and variants thereof.

The components to be cultured in the host cell to package a viral vector in a capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell can contain the required component(s) under the control of an inducible promoter. However, the required component(s) may be under the control of a constitutive promoter. The recombinant viral vector, rep sequences, cap sequences, and helper functions required for producing the viral vector may be delivered to the packaging host cell using any appropriate genetic element. The selected genetic element may be delivered by any suitable method, including those described herein. Other methods are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

In some embodiments, recombinant AAV transfer vectors may be produced using the triple transfection method (e.g., as described in detail in U.S. Pat. No. 6,001,650, U.S. Pat. No. 6,593,123, as well as X. Xiao et al, J. Virol. 72:2224-2232 (1998), and T. Matsushita et al, Gene Ther. 5(7): 938-945 (1998), the contents of which relating to the triple transfection method are incorporated herein by reference). For example, the recombinant AAVs can be produced by transfecting a host cell with a recombinant AAV transfer vector (comprising a transgene) to be packaged into AAV particles, an AAV helper function vector, and an accessory function vector. Generally, an AAV helper function vector encodes AAV helper function sequences (rep and cap), which function in trans for productive AAV replication and encapsidation. Preferably, the AAV helper function vector supports efficient AAV vector production without generating any detectable wild-type AAV virions (i.e., AAV virions containing functional rep and cap genes). The accessory function vector can encode nucleotide sequences for non-AAV derived viral and/or cellular functions upon which AAV is dependent for replication. The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Other methods for producing viral vectors are known in the art. Moreover, viral vectors are available commercially.

In regard to synthetic nanocarriers coupled to immunosuppressants, methods for attaching components to synthetic nanocarriers may be useful.

In embodiments, methods for attaching components to, for example, synthetic nanocarriers may be useful. In certain embodiments, the attaching can be a covalent linker. In embodiments, immunosuppressants according to the invention can be covalently attached to the external surface via a 1,2,3-triazole linker formed by the 1,3-dipolar cycloaddition reaction of azido groups with immunosuppressant containing an alkyne group or by the 1,3-dipolar cycloaddition reaction of alkynes with immunosuppressants containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component such as an immunosuppressant with the carboxylic acid group of a second component such as the nanocarrier. The amide bond in the linker can be made using any of the conventional amide bond forming reactions with suitably protected amino acids and activated carboxylic acid such N-hydroxysuccinimide-activated ester.

A disulfide linker is made via the formation of a disulfide (S—S) bond between two sulfur atoms of the form, for instance, of R1-S—S—R2. A disulfide bond can be formed by thiol exchange of a component containing thiol/mercaptan group (—SH) with another activated thiol group or a component containing thiol/mercaptan groups with a component containing activated thiol group.

A triazole linker, specifically a 1,2,3-triazole of the form

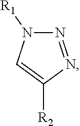

wherein R1 and R2 may be any chemical entities, is made by the 1,3-dipolar cycloaddition reaction of an azide attached to a first component with a terminal alkyne attached to a second component such as the immunosuppressant. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

A thioether linker is made by the formation of a sulfur-carbon (thioether) bond in the form, for instance, of R1-S—R2. Thioether can be made by either alkylation of a thiol/mercaptan (—SH) group on one component with an alkylating group such as halide or epoxide on a second component. Thioether linkers can also be formed by Michael addition of a thiol/mercaptan group on one component to an electron-deficient alkene group on a second component containing a maleimide group or vinyl sulfone group as the Michael acceptor. In another way, thioether linkers can be prepared by the radical thiol-ene reaction of a thiol/mercaptan group on one component with an alkene group on a second component.

A hydrazone linker is made by the reaction of a hydrazide group on one component with an aldehyde/ketone group on the second component.

A hydrazide linker is formed by the reaction of a hydrazine group on one component with a carboxylic acid group on the second component. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component with an aldehyde or ketone group on the second component.

An urea or thiourea linker is prepared by the reaction of an amine group on one component with an isocyanate or thioisocyanate group on the second component.

An amidine linker is prepared by the reaction of an amine group on one component with an imidoester group on the second component.

An amine linker is made by the alkylation reaction of an amine group on one component with an alkylating group such as halide, epoxide, or sulfonate ester group on the second component. Alternatively, an amine linker can also be made by reductive amination of an amine group on one component with an aldehyde or ketone group on the second component with a suitable reducing reagent such as sodium cyanoborohydride or sodium triacetoxyborohydride.

A sulfonamide linker is made by the reaction of an amine group on one component with a sulfonyl halide (such as sulfonyl chloride) group on the second component.

A sulfone linker is made by Michael addition of a nucleophile to a vinyl sulfone. Either the vinyl sulfone or the nucleophile may be on the surface of the nanocarrier or attached to a component.

The component can also be conjugated via non-covalent conjugation methods. For example, a negative charged immunosuppressant can be conjugated to a positive charged component through electrostatic adsorption. A component containing a metal ligand can also be conjugated to a metal complex via a metal-ligand complex.

In embodiments, the component can be attached to a polymer, for example polylactic acid-block-polyethylene glycol, prior to the assembly of a synthetic nanocarrier or the synthetic nanocarrier can be formed with reactive or activatible groups on its surface. In the latter case, the component may be prepared with a group which is compatible with the attachment chemistry that is presented by the synthetic nanocarriers' surface. In other embodiments, a peptide component can be attached to VLPs or liposomes using a suitable linker. A linker is a compound or reagent that capable of coupling two molecules together. In an embodiment, the linker can be a homobifunctional or heterobifunctional reagent as described in Hermanson 2008. For example, a VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a peptide component containing an acid group via the other end of the ADH linker on nanocarrier to produce the corresponding VLP or liposome peptide conjugate.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The component is prepared with the presence of either an alkyne (if the polymer contains an azide) or an azide (if the polymer contains an alkyne) group. The component is then allowed to react with the nanocarrier via the 1,3-dipolar cycloaddition reaction with or without a catalyst which covalently attaches the component to the particle through the 1,4-disubstituted 1,2,3-triazole linker.

If the component is a small molecule it may be of advantage to attach the component to a polymer prior to the assembly of synthetic nanocarriers. In embodiments, it may also be an advantage to prepare the synthetic nanocarriers with surface groups that are used to attach the component to the synthetic nanocarrier through the use of these surface groups rather than attaching the component to a polymer and then using this polymer conjugate in the construction of synthetic nanocarriers.

For detailed descriptions of available conjugation methods, see Hermanson G T “Bioconjugate Techniques”, 2nd Edition Published by Academic Press, Inc., 2008. In addition to covalent attachment the component can be attached by adsorption to a pre-formed synthetic nanocarrier or it can be attached by encapsulation during the formation of the synthetic nanocarrier.

Synthetic nanocarriers may be prepared using a wide variety of methods known in the art. For example, synthetic nanocarriers can be formed by methods such as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

Materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. No. 6,632,671 to Unger issued Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be attached to the synthetic nanocarriers and/or the composition of the polymer matrix.

If synthetic nanocarriers prepared by any of the above methods have a size range outside of the desired range, synthetic nanocarriers can be sized, for example, using a sieve.

Elements of the synthetic nanocarriers may be attached to the overall synthetic nanocarrier, e.g., by one or more covalent bonds, or may be attached by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be attached to components directly or indirectly via non-covalent interactions. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such attachments may be arranged to be on an external surface or an internal surface of a synthetic nanocarrier. In embodiments, encapsulation and/or absorption is a form of attaching.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention may comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, compositions are suspended in sterile saline solution for injection with a preservative.

It is to be understood that the compositions of the invention can be made in any suitable manner, and the invention is in no way limited to compositions that can be produced using the methods described herein. Selection of an appropriate method of manufacture may require attention to the properties of the particular moieties being associated.

In some embodiments, compositions are manufactured under sterile conditions or are terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection.

Administration according to the present invention may be by a variety of routes, including but not limited to subcutaneous, intravenous, intramuscular and intraperitoneal routes. The compositions referred to herein may be manufactured and prepared for administration, in some embodiments coadministration, using conventional methods.

The compositions of the invention can be administered in effective amounts, such as the effective amounts described elsewhere herein. Dosage forms may be administered at a variety of frequencies. In some embodiments of any one of the methods or compositions provided, repeated administration of synthetic nanocarriers comprising an immunosuppressant with or without a viral vector is undertaken.

Aspects of the invention relate to determining a protocol for the methods of administration as provided herein. A protocol can be determined by varying at least the frequency, dosage amount of the synthetic nanocarriers comprising an immunosuppressant and/or viral vector, such as according to the administration regimens provided, and assessing a desired or undesired immune response or transgene expression. A preferred protocol for practice of the invention reduces an immune response against the viral vector or viral antigen thereof and/or promotes transgene expression. The protocol comprises at least the frequency of the administration and doses of the synthetic nanocarriers comprising an immunosuppressant and/or viral vectors, such as according to any one of the administration regimens provided herein. Any one of the methods provided herein can include a step of determining a protocol or the administering steps are performed according to a protocol that was determined to achieve any one or more of the desired results as provided herein.

Another aspect of the disclosure relates to kits. In some embodiments of any one of the kits provided, the kit comprises any one or more of the compositions provided herein. Preferably, the composition(s) is/are in an amount to provide any one or more doses as provided herein. The composition(s) can be in one container or in more than one container in the kit. In some embodiments of any one of the kits provided, the container is a vial or an ampoule. In some embodiments of any one of the kits provided, the composition(s) are in lyophilized form each in a separate container or in the same container, such that they may be reconstituted at a subsequent time. In some embodiments of any one of the kits provided, the kit further comprises instructions for reconstitution, mixing, administration, etc. In some embodiments of any one of the kits provided, the instructions include a description of any one of the methods described herein. Instructions can be in any suitable form, e.g., as a printed insert or a label. In some embodiments of any one of the kits provided herein, the kit further comprises one or more syringes or other device(s) that can deliver the composition(s) in vivo to a subject.

### EXAMPLES

### Example 1: Synthetic Nanocarriers Comprising Rapamycin

### Materials

Rapamycin was purchased from TSZ CHEM (185 Wilson Street, Framingham, Mass. 01702; Product Catalogue # R1017). PLGA with 76% lactide and 24% glycolide content and an inherent viscosity of 0.69 dL/g was purchased from SurModics Pharmaceuticals (756 Tom Martin Drive, Birmingham, Ala. 35211. Product Code 7525 DLG 7A.) PLA-PEG block co-polymer with a PEG block of approximately 5,000 Da and PLA block of approximately 40,000 Da was purchased from SurModics Pharmaceuticals (756 Tom Martin Drive, Birmingham, Ala. 35211; Product Code 100 DL mPEG 5000 5CE). Polyvinyl alcohol (85-89% hydrolyzed) was purchased from EMD Chemicals (Product Number 1.41350.1001).

### Method

Solutions were prepared as follows:

Solution 1: PLGA at 75 mg/mL and PLA-PEG at 25 mg/mL in methylene chloride. The solution was prepared by dissolving PLGA and PLA-PEG in pure methylene chloride.

Solution 2: Rapamycin at 100 mg/mL in methylene chloride. The solution was prepared by dissolving rapamycin in pure methylene chloride.

Solution 3: Polyvinyl alcohol at 50 mg/mL in 100 mM pH 8 phosphate buffer.

An oil-in-water emulsion was used to prepare the nanocarriers. The O/W emulsion was prepared by combining solution 1 (1 mL), solution 2 (0.1 mL), and solution 3 (3 mL) in a small pressure tube and sonicating at 30% amplitude for 60 seconds using a Branson Digital Sonifier 250. The O/W emulsion was added to a beaker containing 70 mM pH 8 phosphate buffer solution (30 mL) and stirred at room temperature for 2 hours to allow the methylene chloride to evaporate and for the nanocarriers to form. A portion of the nanocarriers was washed by transferring the nanocarrier suspension to a centrifuge tube and centrifuging at 75,000×g and 4° C. for 35 min, removing the supernatant, and re-suspending the pellet in phosphate buffered saline. The washing procedure was repeated, and the pellet was re-suspended in phosphate buffered saline for a final nanocarrier dispersion of about 10 mg/mL.

Nanocarrier size was determined by dynamic light scattering. The amount rapamycin in the nanocarrier was determined by HPLC analysis. The total dry-nanocarrier mass per mL of suspension was determined by a gravimetric method.

| Effective Diameter | Rapamycin Content |
|---|---|
| (nm) | (% w/w) |
| 227 | 6.4 |

### Example 2: Synthetic Nanocarriers Comprising GSK1059615

### Materials

GSK1059615 was purchased from MedChem Express (11 Deer Park Drive, Suite 102D Monmouth Junction, N.J. 08852), product code HY-12036. PLGA with a lactide:glycolide ratio of 1:1 and an inherent viscosity of 0.24 dL/g was purchased from Lakeshore Biomaterials (756 Tom Martin Drive, Birmingham, Ala. 35211), product code 5050 DLG 2.5A. PLA-PEG-OMe block co-polymer with a methyl ether terminated PEG block of approximately 5,000 Da and an overall inherent viscosity of 0.26 DL/g was purchased from Lakeshore Biomaterials (756 Tom Martin Drive, Birmingham, Ala. 35211; Product Code 100 DL mPEG 5000 5K-E). Cellgro phosphate buffered saline 1×pH 7.4 (PBS 1×) was purchased from Corning (9345 Discovery Blvd. Manassas, Va. 20109), product code 21-040-CV.

### Method

Solutions were prepared as follows:

Solution 1: PLGA (125 mg), and PLA-PEG-OMe (125 mg), were dissolved in 10 mL of acetone. Solution 2: GSK1059615 was prepared at 10 mg in 1 mL of N-methyl-2-pyrrolidinone (NMP).

Nanocarriers were prepared by combining Solution 1 (4 mL) and Solution 2 (0.25 mL) in a small glass pressure tube and adding the mixture drop wise to a 250 mL round bottom flask containing 20 mL of ultra-pure water under stirring. The flask was mounted onto a rotary evaporation device, and the acetone was removed under reduced pressure. A portion of the nanocarriers was washed by transferring the nanocarrier suspension to centrifuge tubes and centrifuging at 75,600 rcf and 4° C. for 50 minutes, removing the supernatant, and re-suspending the pellet in PBS 1×. The washing procedure was repeated, and the pellet was re-suspended in PBS 1× to achieve a nanocarrier suspension having a nominal concentration of 10 mg/mL on a polymer basis. The washed nanocarrier solution was then filtered using 1.2 μm PES membrane syringe filters from Pall, part number 4656. An identical nanocarrier solution was prepared as above, and pooled with the first after the filtration step. The homogenous suspension was stored frozen at −20° C.

Nanocarrier size was determined by dynamic light scattering. The amount of GSK1059615 in the nanocarrier was determined by UV absorption at 351 nm. The total dry-nanocarrier mass per mL of suspension was determined by a gravimetric method.

| Effective Diameter | GSK1059615 Content |
|---|---|
| (nm) | (% w/w) |
| 143 | 1.02 |

### Example 3: Early AAV-encoded Transgene Expression In Vivo is not Affected if AAV is Premixed with Synthetic Nanocarriers Coupled to Rapamycin

In a standard male mouse AAV transduction model, if AAV is premixed with a synthetic nanocarrier coupled to rapamycin (SVP[Rapa]), in this instance encapsulated rapamycin, early AAV-encoded transgene expression in vivo is not affected; if SVP[Rapa] is administered immediately after AAV, transgene expression is inferior; this effect was found to be independent of IgG antibody formation.

Specifically, groups of 6-12 male C57BL/6 mice were injected (i.v., tail vein) with AAV-SEAP with or without SVP-encapsulated rapamycin (SVP[Rapa] in this example), which was either admixed to AAV and then administered or was injected immediately after AAV-SEAP (within 15 min interval; labelled as ‘not admixed’). At time indicated (day 19) mice were bled, serum separated from whole blood and stored at −20±5° C. until analysis. Then SEAP levels in serum were measured using an assay kit from ThermoFisher Scientific (Waltham, Mass., USA). Briefly, sera samples and positive controls were diluted in dilution buffer, incubated at 65° C. for 30 min, then cooled to room temperature, plated into 96-well format, assay buffer (5 min) and then substrate (20 min) added and plates read on luminometer (477 nm).

Separately, IgG antibody to AAV was measured in an ELISA assay: 96-well plates coated overnight with the AAV, washed and blocked on the following day, then diluted serum samples (1:40) added to the plate and incubated; plates then washed, goat anti-mouse IgG specific-HRP added and after another incubation and wash, the presence of IgG antibodies to AAV detected by adding TMB substrate and measuring at an absorbance of 450 nm with a reference wavelength of 570 nm (the intensity of the signal presented as top optical density, OD, is directly proportional to the quantity of IgG antibody in the sample).

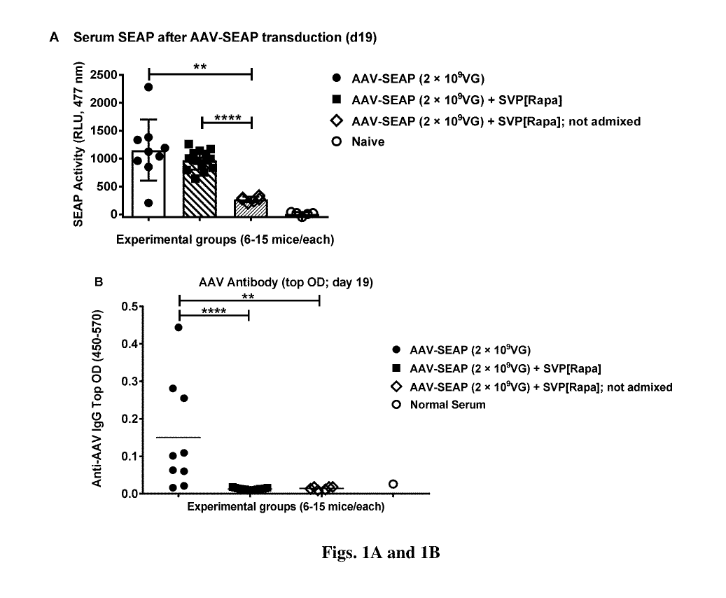

While admixed SVP[Rapa] did not affect SEAP expression at this time point, SEAP expression was downregulated in mice sequentially injected with AAV-SEAP followed by SVP[Rapa] (FIG. 1A). This effect was independent of induction of IgG antibody to AAV since at this point all mice treated with SVP[Rapa] demonstrated downregulation of IgG antibody to AAV (FIG. 1B).

### Example 4: Non-Admixed Synthetic Nanocarriers Coupled to Rapamycin Results in Early Downregulation of AAV-Driven Transgene Expression Irrespective of Order of Administration

In this experiment it was found that non-admixed SVP[Rapa] results in early downregulation of AAV-driven transgene expression irrespective of the order of administration. It was found that transgene expression levels increase with time in mice that received AAV combined with SVP[Rapa]; this effect is not related to IgG antibody downregulation by SVP[Rapa].

Specifically, groups of 5-6 male C57BL/6 mice were injected i.v. with AAV-SEAP with or without SVP[Rapa], which was either admixed to AAV or was injected separately before or after AAV-SEAP with a 15-min or 1-hr interval. At times indicated (d19 and d75) SEAP activity and IgG antibodies to AAV in mouse sera were measured.

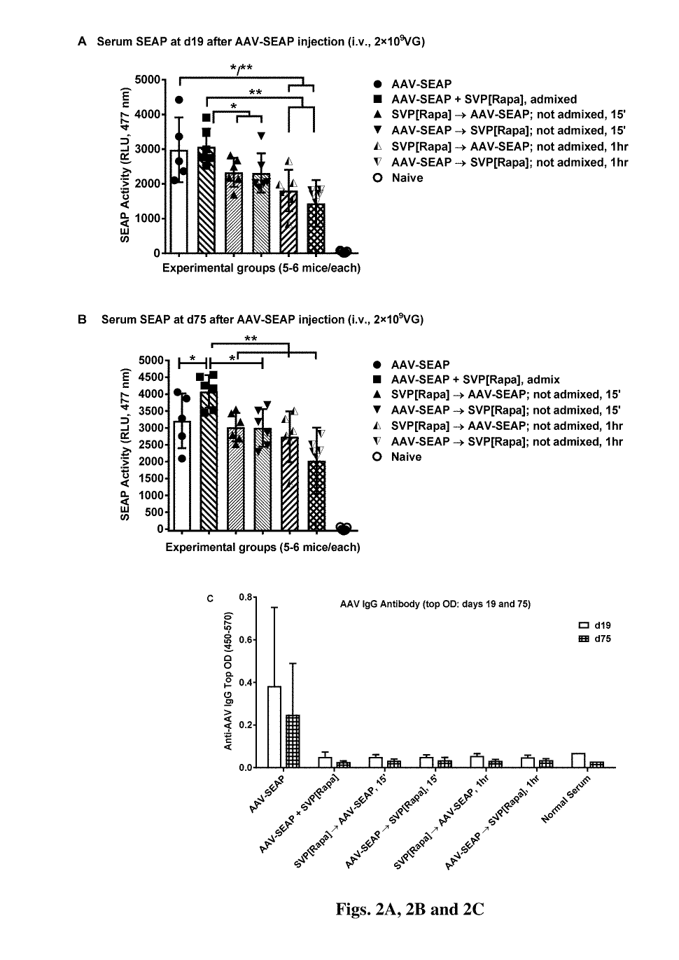

Separate administration of AAV-SEAP and SVP[Rapa] led to lower expression of SEAP at day 19 (FIG. 2A). Mice treated with a 1-hr interval showed somewhat lower expression than those injected with a 15-min interval. Mice administered with admixed AAV-SEAP and SVP[Rapa] had the same levels of SEAP expression as those injected with AAV-SEAP alone (FIG. 2A). Notably, levels of SEAP expression grew with time in all mice that received SVP[Rapa] and by day 75 those mice that received admixed AAV-SEAP and SVP[Rapa] expressed SEAP to higher levels than those receiving AAV-SEAP only, while there were groups of mice that had received non-admixed AAV-SEAP and SVP[Rapa] that produced SEAP levels similar to those that received AAV-SEAP only (FIG. 2B). This phenomenon was independent of IgG antibody downregulation, which was seen in all groups, which received SVP[Rapa] (FIG. 2C).

### Example 5: Admixed Synthetic Nanocarriers Coupled to Rapamycin and AAV-SEAP Leads to Immediate Elevation of Transgene Expression Irrespective of IgG Antibody Response

In this experiment, it was found that administration of SVP[Rapa] with AAV-SEAP to female mice leads to immediate elevation of transgene expression irrespective of IgG antibody response.

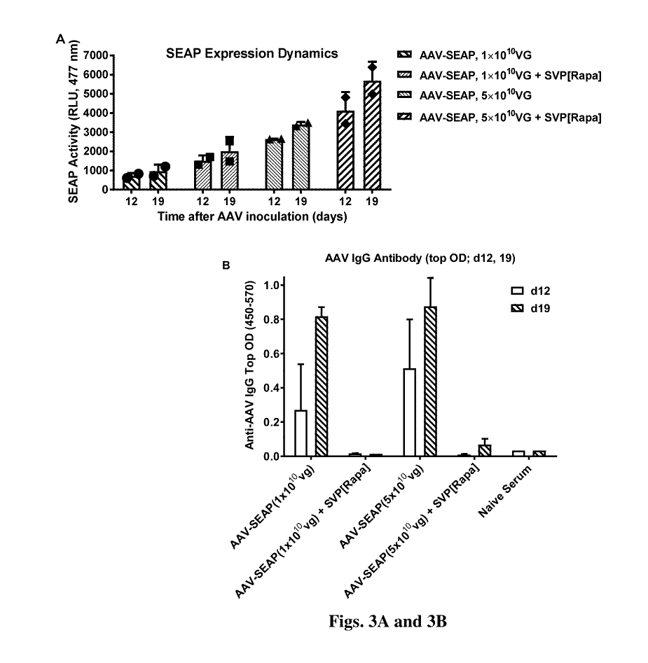

From Examples 3 and 4, it appears that non-admixing SVP[Rapa] and AAV may have inferior effects in the short term. However, the phenomenon may be masked at an early time-point (such as day 19) by efficient transduction by AAV that is commonly seen in male C57BL/6 mice. Separately, groups of C57BL/6 female mice were inoculated i.v. with two different doses of AAV-SEAP with or without SVP[Rapa] with SEAP activity and AAV IgG antibodies measured in sera at days 12 and 19. It was found that elevated levels of SEAP expression occur immediately after AAV inoculation in all mice that received admix of AAV-SEAP and SVP[Rapa] with an average two-fold improvement (FIG. 3A). Notably, this was observed at a very early time-point such as day 12, at which minimal IgG antibody induction is seen (FIG. 3B). In addition, the relative levels of SEAP expression between the groups stayed the same within the given time interval (between 12 and 19 days after injection), while IgG antibody levels in SVP[Rapa]-untreated groups grew over the same time (FIG. 3B).

These results, confirm that administration of transgene-carrying AAV with SVP[Rapa] leads to higher levels of transgene expression in vivo, which is especially noticeable in systems less amenable to AAV transduction and that this phenomenon is independent of AAV IgG antibody downregulation by SVP[Rapa].

### Example 6: Admixing of AAV and Synthetic Nanocarriers Coupled to Rapamycin In Vitro Leads to its Full Adsorption within 15 Minutes

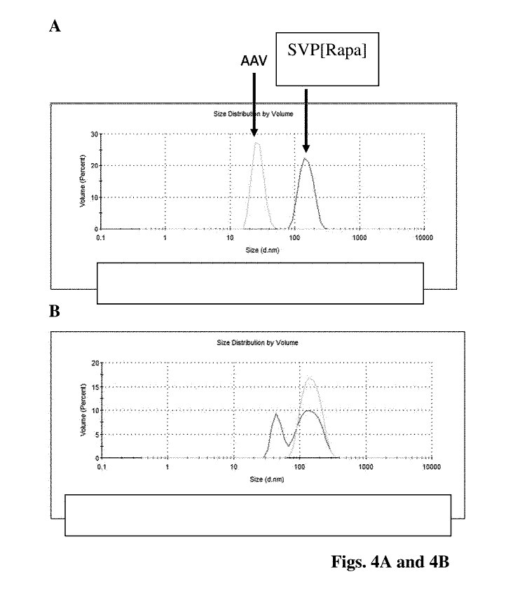

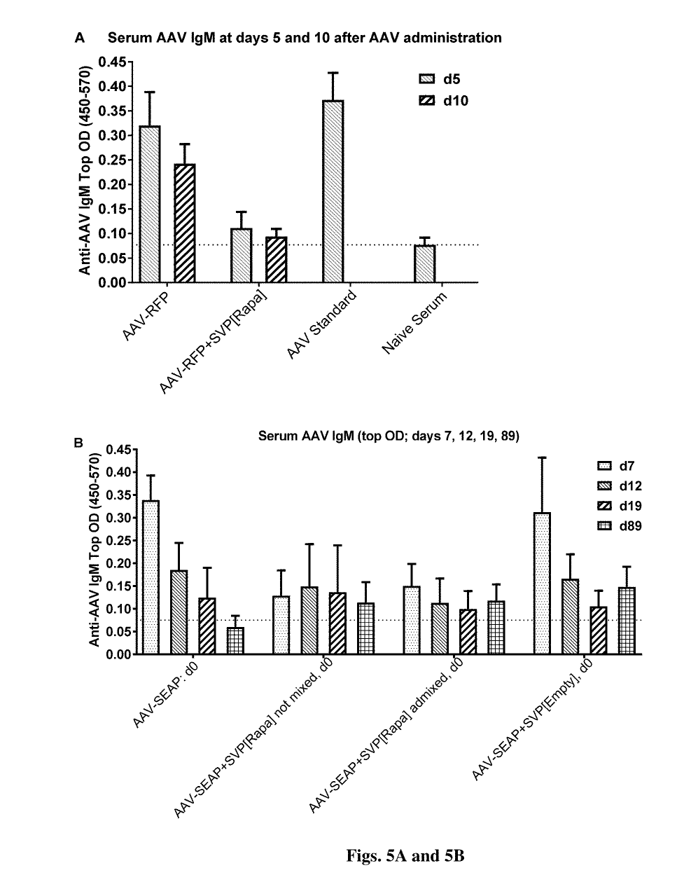

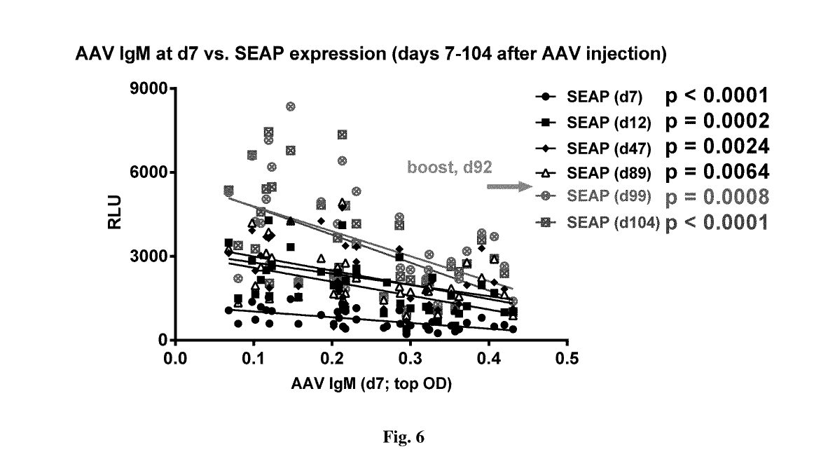

Specifically, 2.5×10¹¹ VG of AAV in 1 mL of PBS or SVP[Rapa] particles were added to a quartz cuvette and measured by DLS separately (FIG. 4A) or after admixing (at AAV to SVP[Rapa] particle ratio of 100:1) either immediately or after 15-minute incubation (FIG. 4B).

Immediately after admixing of AAV to SVP[Rapa] (FIG. 4B) two separate peaks were observed which corresponded to sizes of AAV and SVP[Rapa] measured separately (FIG. 4A; 25 and 150 nm, correspondingly). At 15 minutes after admixing of SVP[Rapa] to AAV only a single peak was observed (FIG. 4B), which corresponded to the size of nanocarrier indicating a full adsorption of AAV to SVP[Rapa].

### Example 7: Early AAV I₂M Induction is Downregulated by Administration of Synthetic Nanocarriers Coupled to Rapamycin and Viral Vector

Groups of 5 female C57BL/6 mice were injected (i.v., tail vein) with 1×10¹⁰ viral genomes (VG) AAV-SEAP with or without SVP-encapsulated rapamycin (SVP[Rapa] in this example) or control polymer-only (SVP[Empty] in this example), which was either admixed to AAV and then administered or was injected immediately prior to AAV-SEAP (within 15 min interval; labelled as ‘not admixed’). At times indicated (days 5 and 10 in A and days 6, 12, 19 and 89 in B) mice were bled, serum separated from whole blood and stored at −20±5° C. until analysis. Separately, IgM antibody to AAV was measured with an ELISA assay: 96-well plates coated overnight with the AAV, washed and blocked on the following day, then diluted serum samples (1:40) added to the plate and incubated; plates then washed, goat anti-mouse IgM specific-HRP added and after another incubation and wash, the presence of IgM antibodies to AAV detected by adding TMB substrate and measuring at an absorbance of 450 nm with a reference wavelength of 570 nm (the intensity of the signal presented as top optical density, OD, is directly proportional to the quantity of IgM antibody in the sample).

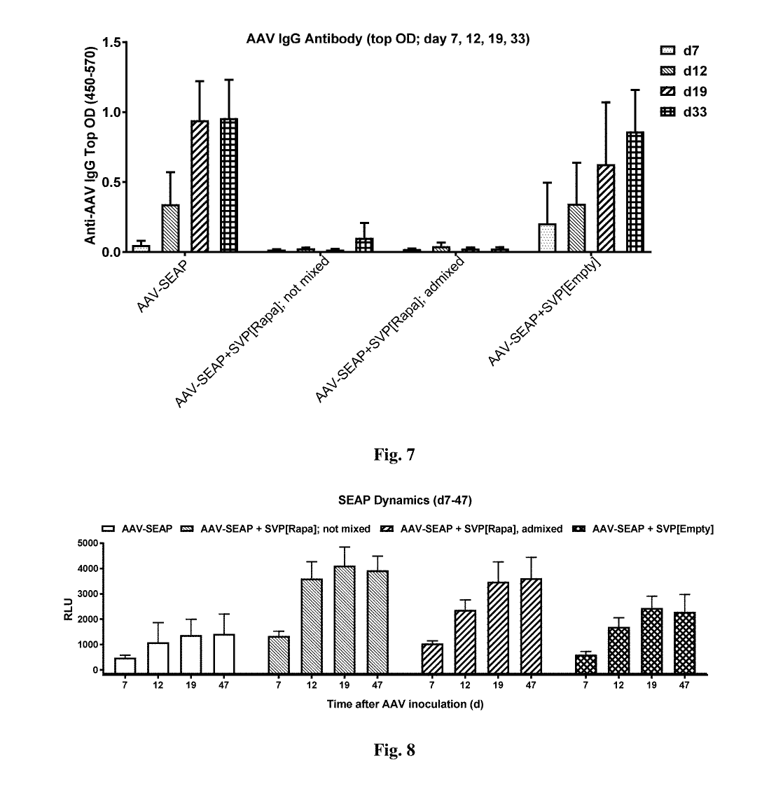

Both admixed and non-admixed AAV administered SVP[Rapa] strongly downregulated early induction of IgM at days 5 (FIG. 5A) and 7 (FIG. 5B) after AAV injection to the levels close to normal serum baseline (dashed lines). This effect was still observed at day 10 (FIG. 5A), but less pronounced by day 12 and further (FIG. 5B), at which point levels of IgM in the untreated mice tapered down. There was no IgM downregulating activity seen in the group treated with control SVP[Empty] nanocarrier.

### Example 8: Levels of Early IgM Against AAV Capsid Inversely Correlate with Levels of Transgene Expression after AAV Administration

Eight groups of 4-5 female C57BL/6 mice were injected i.v. with AAV-SEAP (1×10¹⁰ VG) with or without SVP[Rapa] or with SVP[Empty], which was either admixed to AAV or was injected separately immediately before AAV-SEAP. At times indicated (d7 to d89) SEAP activity and AAV IgM levels were measured (FIG. 6). On day 92 all animals were boosted with the same amounts of AAV-SEAP and subjected to the same treatments as at prime. SEAP levels in serum were measured using an assay kit from ThermoFisher Scientific (Waltham, Mass., USA). Briefly, sera samples and positive controls were diluted in dilution buffer, incubated at 65° C. for 30 min, then cooled to room temperature, plated into 96-well format, assay buffer (5 min) and then substrate (20 min) added and plates read on luminometer (477 nm).

IgM levels on d7 showed an extremely strong and statistically significant inverse correlation with serum SEAP levels (p values indicated on the graph) at day 7 after AAV administration, when the overall levels of SEAP in serum are generally low. This correlation was maintained for nearly three months after initial AAV and SVP[Rapa] administration. Moreover, after AAV-SEAP boost at day 92 those animals which initially had low levels of AAV IgM responded to the boost in a more beneficial manner, i.e. by elevating transgene expression to higher levels, while those animals with initially high IgM levels responded in a weaker fashion, i.e., by a lower elevation of transgene expression. As a result, the inverse correlation between initial (day 7) AAV IgM levels and post-boost serum SEAP levels became even stronger after the boost (d99 and d104 or days 7 and 12 post boost).

### Example 9: Administration of Synthetic Nanocarriers Coupled to Rapamycin Prior to the Synthetic Nanocarriers and a Viral Vector (Prophetic)

A group of subjects are injected i.v. with SVP[Rapa], and within 30 days the subjects are injected i.v. with AAV-SEAP (1×10¹⁰ VG) with SVP[Rapa], which is either admixed or not admixed but administered simultaneously. At times indicated SEAP activity and AAV IgM levels are measured.

### Example 10: Further Administration of Synthetic Nanocarriers Coupled to Rapamycin and a Viral Vector (Prophetic)

Within 30 days of the second administration of the subjects of Example 9, the subjects are again injected i.v. with SVP[Rapa]. Within another 30 days, the subjects are injected i.v. with AAV-SEAP (1×10¹⁰ VG) with SVP[Rapa], which is either admixed or not admixed but administered simultaneously. At times indicated SEAP activity and AAV IgM levels are again measured.

### Example 11: IgG Suppression

Groups of 5 female C57BL/6 mice were injected (i.v., tail vein) with 1×10¹⁰ viral genomes (VG) AAV-SEAP alone or with SVP-encapsulated rapamycin, in this example (SVP[Rapa]), or control polymer-only, in this example, (SVP[Empty]), with the former being either admixed to AAV and then administered or injected prior to AAV-SEAP (within 15 minutes; labelled as ‘not admixed’). At times indicated mice were bled, serum separated from whole blood and stored at −20±5° C. until analysis.

IgG antibody to AAV was measured in an ELISA assay: 96-well plates coated overnight with the AAV, washed and blocked on the following day, then diluted serum samples (1:40) added to the plate and incubated; plates then washed, goat anti-mouse IgG specific-HRP added and after another incubation and wash, the presence of IgG antibodies to AAV detected by adding TMB substrate and measuring at an absorbance of 450 nm with a reference wavelength of 570 nm (the intensity of the signal presented as top optical density, OD, is proportional to the quantity of IgG antibody in the sample). SEAP levels were measured using an assay kit from ThermoFisher Scientific (Waltham, Mass., USA). Sera samples and positive controls were diluted in dilution buffer, incubated at 65° C. for 30 min, cooled to room temperature, plated into 96-well format, assay buffer (5 min) and then substrate (20 min) added and plates read on luminometer (477 nm).

Both admixed and non-admixed SVP[Rapa] suppressed early induction of IgG to AAV (FIG. 7). This effect was strong irrespective of whether SVP[Rapa] was admixed to AAV or administered separately prior to AAV injection.

Both AAV-admixed and non-admixed SVP[Rapa] promoted early and consistent elevation of SEAP expression in serum (FIG. 8). SEAP expression in both SVP[Rapa]-treated groups was higher than that in the untreated group by a factor of 2.5-3.0 and also than in the group treated with a control SVP[Empty]. This difference was seen at day 7 and persisted for at least 7 weeks.

### Example 12: IgM and IgG Suppression

Groups of 5 female C57BL/6 mice were injected (i.v., tail vein) with 1×10¹⁰ viral genomes (VG) AAV alone or with SVP-encapsulated rapamycin (SVP[Rapa] in this example) either admixed to AAV and then administered (day 0), injected separately at one day prior to AAV (day −1), or both injected separately at one day prior to AAV and admixed (days −1, 0). At times indicated mice were bled, serum separated from whole blood and stored at −20±5° C. until analysis. Levels of IgM and IgG against AAV were determined as described above.

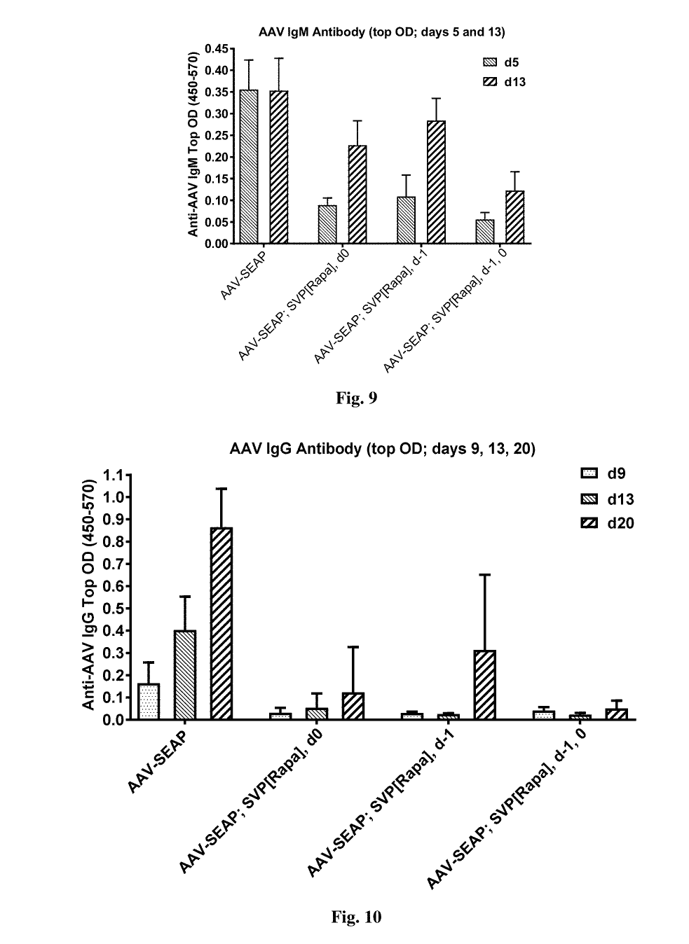

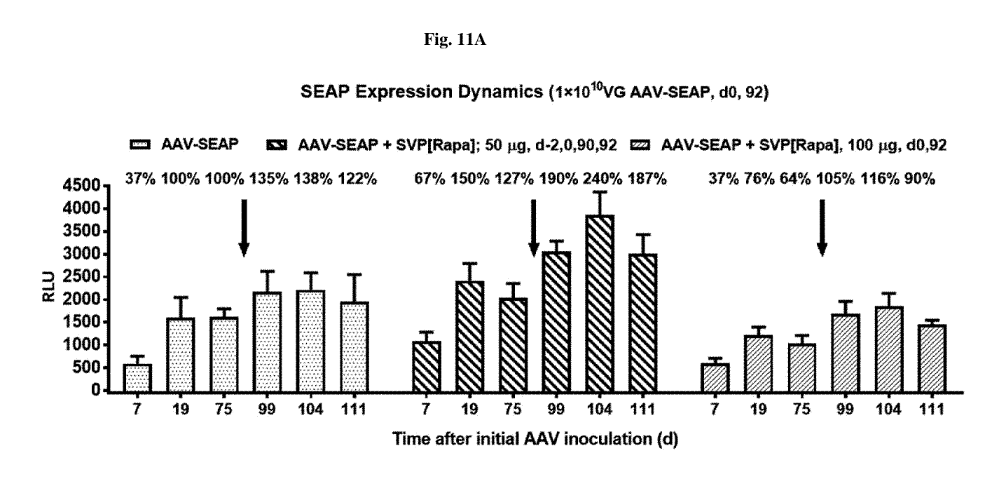

While SVP[Rapa] admixed with AAV led to suppression of both AAV IgM (FIG. 9) and IgG (FIG. 10), a similar effect was seen if SVP[Rapa] was administered separately from AAV one day earlier. Notably, both AAV IgM (by day 13, FIG. 9) and IgG (by day 20, FIG. 10) in these two groups started to become elevated at later time-points, although their levels stayed lower than in untreated mice. At the same time, mice treated with SVP[Rapa] at one day prior to AAV injection and also admixed (d −1, 0) showed the lowest AAV IgM levels at day 5 (with marginal elevation by day 13, FIG. 9) and no AAV IgG development up to day 20 (FIG. 10). Thus, production of AAV IgM and IgG antibodies was suppressed more strongly in mice receiving SVP[Rapa] treatments on day −1 and day 0.

### Example 13: Synthetic Nanocarriers Comprising an Immunosuppressant

Synthetic nanocarriers comprising an immunosuppressant, such as rapamycin, can be produced using any method known to those of ordinary skill in the art. Preferably, in some embodiments of any one of the methods or compositions provided herein the synthetic nanocarriers comprising an immunosuppressant are produced by any one of the methods of US Publication No. US 2016/0128986 A1 and US Publication No. US 2016/0128987 A1, the described methods of such production and the resulting synthetic nanocarriers being incorporated herein by reference in their entirety. In any one of the methods or compositions provided herein, the synthetic nanocarriers comprising an immunosuppressant are such incorporated synthetic nanocarriers. Synthetic nanocarriers comprising rapamycin were produced with methods at least similar to these incorporated methods and used in the following Examples.

### Example 14: Split Doses of Synthetic Nanocarriers Comprising an Immunosuppressant

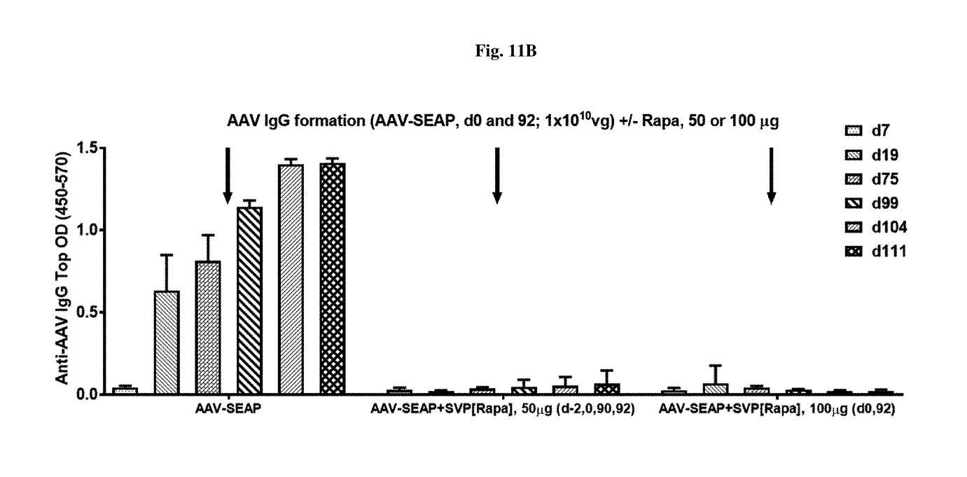

Splitting doses of rapamycin, when comprised in synthetic nanocarriers, into two parts and administering the first half prior to AAV vector co-injection with the second half of the rapamycin, when comprised in synthetic nanocarriers, dose was found to be beneficial, both in terms of transgene expression (FIG. 11A) and for its suppressive effect on antiviral IgG (FIG. 11B), relative to the same cumulative dose of rapamycin, when comprised in synthetic nanocarriers, co-injected with the AAV vector.

Groups of 5 female C57BL/6 mice were injected on days 0 and 92 (intravenous, i.v., tail vein) with 1×10¹⁰ viral genomes (VG) of AAV-SEAP either alone (AAV-SEAP) or with rapamycin-comprising synthetic nanocarriers (AAV-SEAP+rapamycin-comprising synthetic nanocarriers, 100 μg, d0, 92) or with rapamycin-comprising synthetic nanocarriers (50 μg rapamycin) delivered two days prior to AAV injection and with AAV injection (AAV-SEAP+rapamycin-comprising synthetic nanocarriers, d-2, 0, 90, 92). At the times indicated in FIG. 11A (days 7, 19, 75, 99, 104 and 111), mice were bled, and the serum was separated from the whole blood and stored at −20±5° C. until analysis.

SEAP levels in serum were measured using an assay kit from ThermoFisher Scientific (Waltham, Mass., USA). Briefly, sera samples and positive controls were diluted in dilution buffer, incubated at 65° C. for 30 minutes (min), then cooled to room temperature, plated into a 96-well format, incubated with assay buffer (5 min), and then substrate added (20 min) and the plates were read using a luminometer (477 nm).

Separately, IgG antibody to AAV was measured using an ELISA. 96-well plates were coated overnight with the AAV, and then washed and blocked on the following day. Diluted serum samples (1:40) were added to the plate and incubated. The plates were then washed, and goat anti-mouse IgG specific-HRP was added. After another incubation and wash, the presence of IgG antibodies to AAV was detected by adding TMB substrate and measuring at an absorbance of 450 nm with a reference wavelength of 570 nm (the intensity of the signal presented as top optical density, OD, is directly proportional to the quantity of IgG antibody in the sample in FIG. 11B).

Administration of rapamycin-comprising synthetic nanocarriers (50 μg) 2 days prior to co-administration of rapamycin-comprising synthetic nanocarriers (50 μg) admixed to AAV-SEAP led to immediate elevation of SEAP expression (FIG. 11A), which at certain time-points was nearly two times higher than without SVP. Relative expression is shown for each time-point in each group above the graph compared to that in untreated mice at day 19 (d19) (100%). At the same time, the same total 100 μg dose (rapamycin-comprising synthetic nanocarriers admixed and co-administered with AAV) did not have a beneficial effect on transgene expression. A similar effect was seen after the day 92 boost (indicated by an arrow). Notably, both regimens of administration of rapamycin-comprising synthetic nanocarriers equally suppressed the formation of an IgG response to AAV after prime and boost (FIG. 11B).

### Example 15: Dosing of Synthetic Nanocarriers Comprising an Immunosuppressant in at Least Two Parts

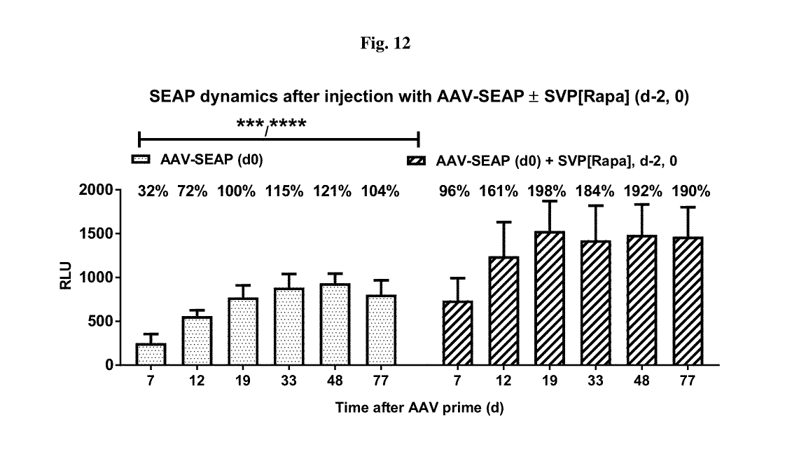

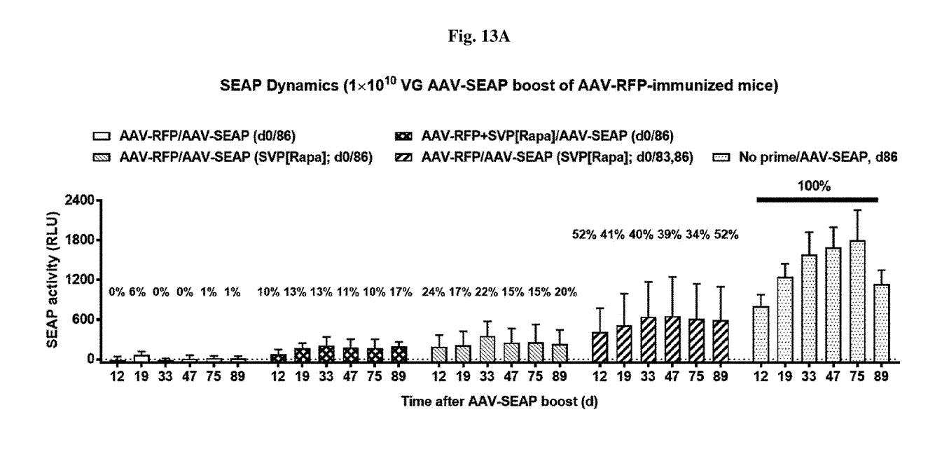

The delivery of the rapamycin, when comprised in synthetic nanocarriers, dose in two parts, with the first part being administered two days prior to the AAV co-injection with the second half of the dose, was found to lead to stably elevated transgene expression (FIG. 12).

Groups of 9-10 female C57BL/6 mice were injected on day 0 (i.v., tail vein) with 1×10¹⁰ VG of AAV-SEAP either alone (AAV-SEAP) or with rapamycin, when comprised in synthetic nanocarriers, at 50 μg delivered two days prior to AAV injection and with AAV injection (AAV-SEAP+rapamycin-comprising synthetic nanocarriers, d-2, 0). At times indicated (days 7, 12, 19, 33, 48, and 77) mice were bled, and the serum was separated from whole blood and stored at −20±5° C. until analysis. SEAP levels in serum were measured as described in Example 14.

Administration of 50 μg of rapamycin, when comprised in synthetic nanocarriers, 2 days prior to co-administration of another 50 μg of the rapamycin, when comprised in synthetic nanocarriers, admixed to AAV-SEAP led to an immediate elevation of SEAP expression, which generally was 2 times higher than without synthetic nanocarriers (and was three times higher early, 7 days after AAV administration). This difference was stable and maintained at all consecutive time-points (relative expression is shown for each time-point in each group above the graph compared to that in untreated mice at d19 taken as 100%).

### Example 16: Additional Dose of Synthetic Nanocarriers Comprising an Immunosuppressant

The delivery of an additional dose of rapamycin, when comprised in synthetic nanocarriers, prior to AAV vector co-injection with rapamycin, when comprised in synthetic nanocarriers, into AAV-immune mice was found to lead to elevated transgene expression.

Since pre-administration of the 50 μg of rapamycin, when comprised in synthetic nanocarriers, was shown to be beneficial for transgene expression after AAV prime, whether it is also beneficial in animals previously exposed to AAV was examined. Groups of 5 female C57BL/6 mice were injected on day 0 (i.v., tail vein) with 1×10¹⁰ VG of AAV-RFP either alone or admixed with rapamycin, when comprised in synthetic nanocarriers, at 50 μg and then boosted with the same dose of AAV-SEAP alone or admixed with rapamycin, when comprised in synthetic nanocarriers, or with rapamycin, when comprised in synthetic nanocarriers, both admixed to AAV-SEAP and pre-injected at three days prior to AAV-SEAP. At the times indicated, the mice were bled, and the serum was separated from whole blood and stored at −20±5° C. until analysis. SEAP levels in serum and IgG to AAV were measured as described in Example 14.

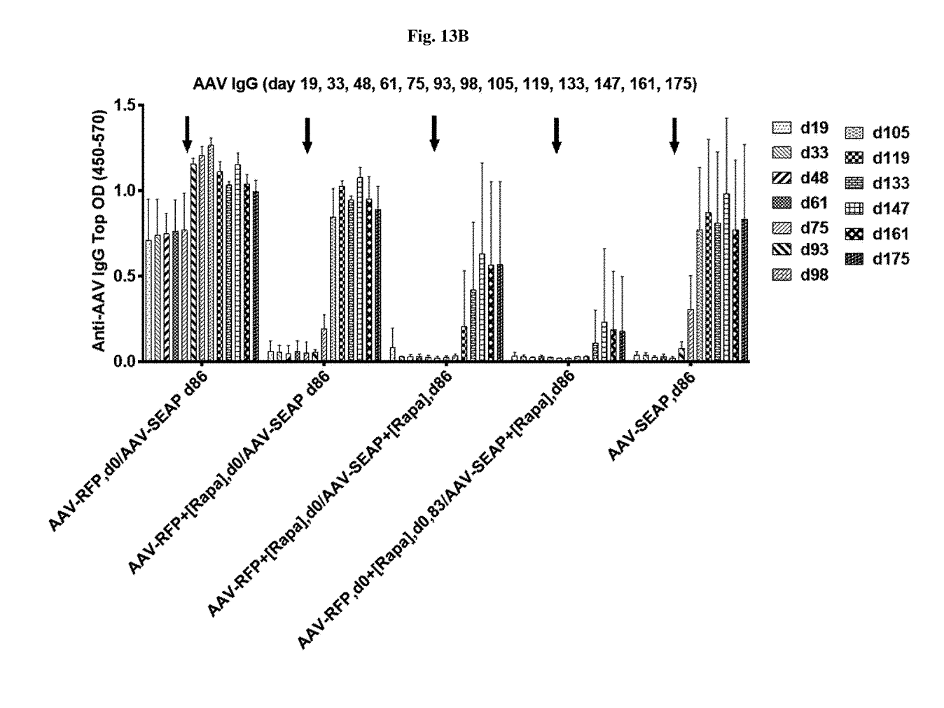

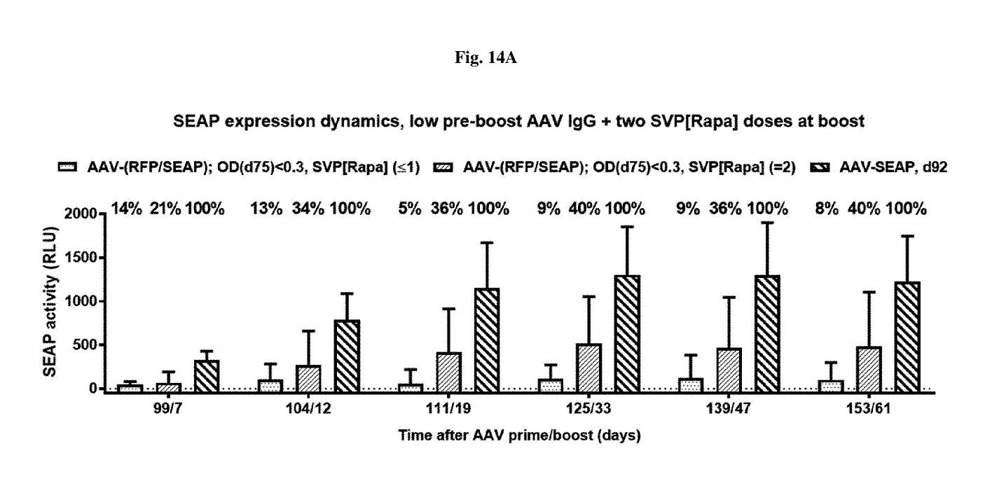

Animals not treated with rapamycin-comprising synthetic nanocarriers (AAV-RFP/AAV-SEAP) showed no meaningful SEAP transgene expression (FIG. 13A). Administration of 50 μg of rapamycin, when comprised in synthetic nanocarriers, at AAV-RFP prime only (AAV-RFP+rapamycin-comprising synthetic nanocarriers/AAV-SEAP) showed low levels of transgene expression (generally, 10-13% from that of naïve mice not pre-injected with AAV-RFP). Further elevation of transgene expression was attained by rapamycin-comprising synthetic nanocarrier administration both at prime and boost (AAV-RFP/AAV-SEAP; rapamycin-comprising synthetic nanocarriers, d0, 86) which sometimes exceeded 20% and stayed within a 15-24% interval. In contrast, additional rapamycin-comprising synthetic nanocarrier administration at 3 days prior to AAV boost led to much higher elevation of SEAP expression, which sometimes exceeded 50% from that of naïve mice and stayed within a 34-52% range (relative expression is shown for each time-point in each group above the graph compared to that in non-primed mice at every time-point taken as 100%).

This transgene expression ranking closely and inversely corresponded to the presence of AAV IgG with mice untreated with rapamycin-comprising synthetic nanocarriers showing immediate IgG production, which was then further elevated by boost (shown by arrows in FIG. 13B). Mice treated with rapamycin-comprising synthetic nanocarriers at prime only developed AAV IgG soon after the boost, while those treated at both prime and boost showed post-boost antibody development delayed by several weeks. Notably, mice additionally treated with rapamycin-comprising synthetic nanocarriers prior to AAV boost mostly stayed antibody-negative for the duration of the study, with only a single mouse showing detectable IgG antibody at 7 weeks after the boost (FIG. 13B).

### Example 17: Additional Doses of Synthetic Nanocarriers Comprising an Immunosuppressant

The delivery of additional doses of rapamycin-comprising synthetic nanocarriers prior to AAV vector and rapamycin-comprising synthetic nanocarrier co-injection into mice with low pre-existing levels of AAV IgG (and not treated with rapamycin-comprising synthetic nanocarriers at the initial priming dose) was found to be essential to post-boost transgene expression.

Since the pre-administration of an additional 50 μg of rapamycin, when comprised in synthetic nanocarriers, was shown to be beneficial for transgene expression after AAV boost in animals previously exposed to AAV, but also treated with rapamycin-comprising synthetic nanocarriers at initial prime, whether similar benefit was found in AAV-pre-exposed animals that were immunized by AAV without rapamycin-comprising synthetic nanocarrier co-administration was examined. Groups of 5-7 female C57BL/6 mice were injected on day 0 (i.v., tail vein) with 2×10⁹ VG of AAV-RFP, then those mice with low levels of AAV IgG (top OD at day 75 post-prime <0.3) were selected and boosted on day 92 with 1×10¹⁰ VG of AAV-SEAP alone or admixed with rapamycin-comprising synthetic nanocarriers or with rapamycin-comprising synthetic nanocarriers both admixed to AAV-SEAP and pre-injected at two days prior to AAV-SEAP. At the times indicated, the mice were bled, and the serum was separated from whole blood and stored at −20±5° C. until analysis. SEAP levels in serum and IgG to AAV were measured as described in Example 14.

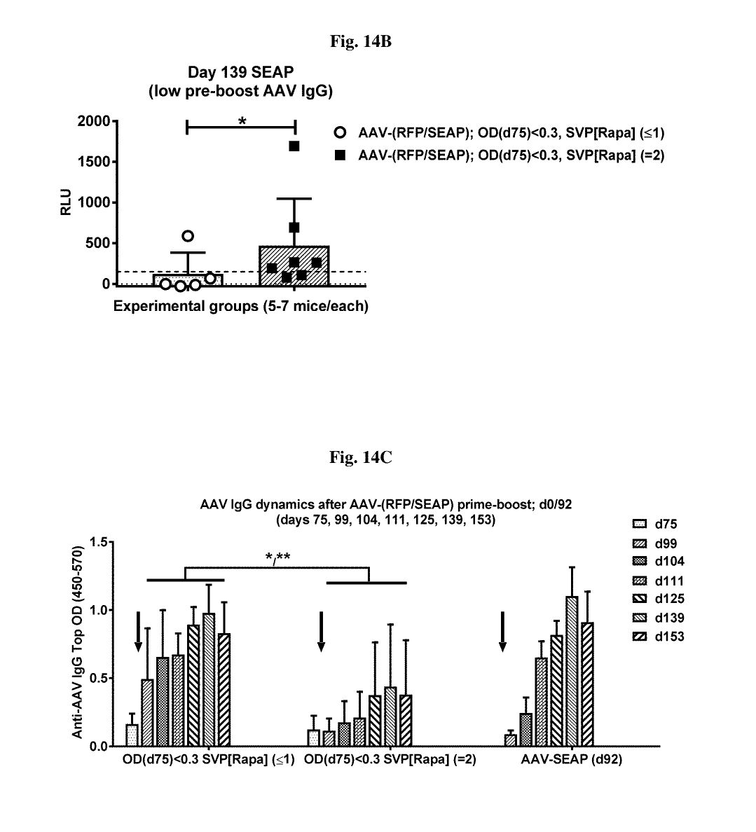

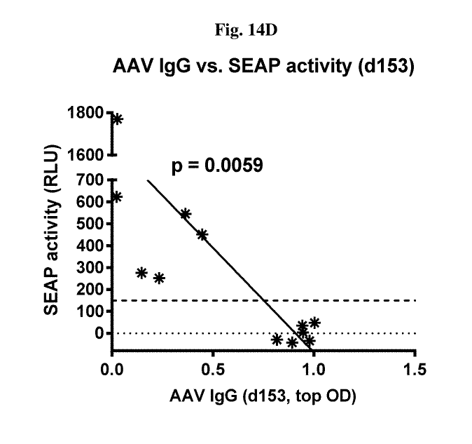

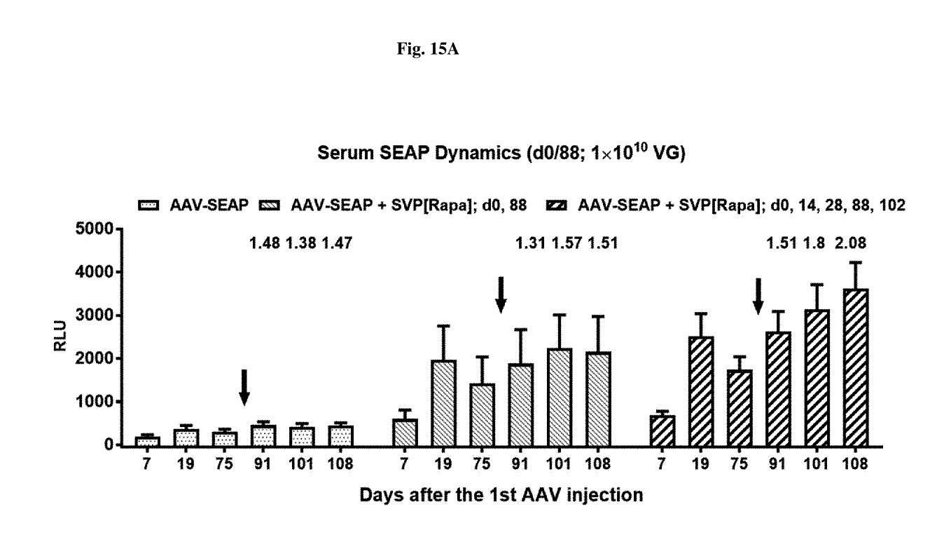

Animals not treated with rapamycin-comprising synthetic nanocarriers or receiving a single rapamycin-comprising synthetic nanocarrier administration at boost (AAV-RFP/SEAP; rapamycin-comprising synthetic nanocarriers ≤1) showed very little SEAP transgene expression (FIG. 14A). Transgene expression was usually within the 5-9% interval (as compared to expression in naïve mice at 100%) and was due to a single mouse out of five demonstrating a meaningful expression level (see left column in FIG. 14B). In comparison, mice from the group that was administered 50 μg of rapamycin, when comprised in synthetic nanocarriers, 2 days prior to AAV boost and also at boost (AAV-RFP/SEAP; rapamycin-comprising synthetic nanocarriers=2) showed much more pronounced SEAP expression, which generally stayed within the 34-40% range compared to that of naïve mice (relative expression is shown in FIG. 14A for each time-point in each group). Notably, five out of seven mice in this group showed detectable SEAP expression (see right column in FIG. 14B), which led to statistically significant different levels of SEAP expression between the experimental groups (FIG. 14B).

This post-boost transgene expression activity in AAV-immune mice closely and inversely corresponded to the development of anamnestic response to AAV as demonstrated by the elevation of AAV IgG in mice receiving less than two treatments with rapamycin-comprising synthetic nanocarriers and a stifling of this response in mice which received two rapamycin-comprising synthetic nanocarrier treatments prior to and at AAV boost (FIG. 14C, boost is shown by arrows). Mice that were treated with less than two doses of rapamycin-comprising synthetic nanocarriers showed a strong AAV IgG booster response as early as 7 days after boost (FIG. 14C), as all but one mouse out of five became strongly AAV IgG-positive. At the same, mice treated with rapamycin-comprising synthetic nanocarriers twice showed much lower AAV anamnestic antibody response with it, becoming statistically different as early as 7 days after the day 92 boost (d99 in FIG. 14C), as only two mice out of seven became strongly AAV IgG-positive. Notably, antibody levels in this group were consistently lower than in naïve mice which were exposed to AAV for the first time at day 92 (reference control group in FIGS. 14A and 14C). Not surprisingly, exactly these mice (one in the group receiving less than two rapamycin-comprising synthetic nanocarrier treatments and five in the group receiving two rapamycin-comprising synthetic nanocarrier treatments) were the same that consistently showed a meaningful SEAP expression resulting in a statistically significant inverse correlation between AAV IgG and serum SEAP levels in the two experimental groups (FIG. 14D).

### Example 18: Dosing of Synthetic Nanocarriers Comprising an Immunosuppressant and Viral Vector

Rapamycin-comprising synthetic nanocarrier doses administered after AAV vector and rapamycin-comprising synthetic nanocarrier co-injection were found to provide additional benefit for transgene expression and AAV antibody suppression.

Although the co-administration of rapamycin-comprising synthetic nanocarriers with AAV was shown to provide immediate benefits for AAV-driven transgene expression and to effectively suppress antibodies to AAV, whether further rapamycin-comprising synthetic nanocarrier injections would provide for a further benefit was examined. Groups of 5 female C57BL/6 mice were injected on days 0 and 88 (i.v., tail vein) with 1×10¹⁰ VG of AAV-SEAP either alone or admixed with rapamycin, when comprised in synthetic nanocarriers, at 50 μg and one group was then treated with two additional bi-weekly rapamycin-comprising synthetic nanocarrier injections both after prime and boost (d14, 28, 102 and 116). At the times indicated, mice were bled, and the serum was separated from whole blood and stored at −20±5° C. until analysis. SEAP levels in serum and IgG to AAV were measured as described in Example 14.

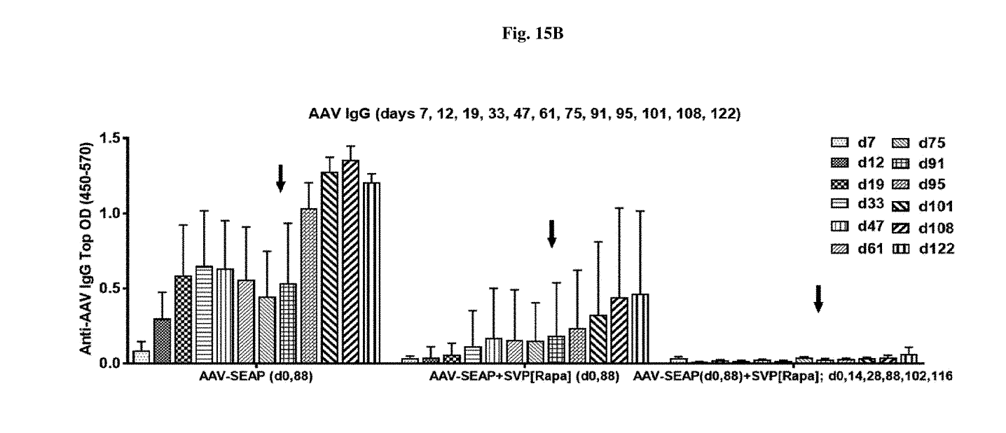

As shown earlier, the administration of 50 μg of rapamycin, when comprised in synthetic nanocarriers, admixed to AAV-SEAP led to an immediate elevation of SEAP expression (FIG. 15A), which at certain time-points was 4 times higher than the group without the synthetic nanocarriers. However, additional rapamycin-comprising synthetic nanocarrier treatments provided even more pronounced benefits, with resulting expression levels being 6-7-fold higher than in untreated mice. Even further elevation was seen after the day 88 boost (indicated by an arrow; relative expression is shown for each post-boost time-point compared to pre-boost d75 SEAP levels in each group). While rapamycin-comprising synthetic nanocarrier administration at boost provided a modest additional benefit with resulting transgene expression stabilizing at a 5-fold excess compared to untreated mice, the benefit of additional rapamycin-comprising synthetic nanocarrier treatments continued to elevate up to an 8-fold differential at day 108. This corresponded to the more pronounced AAV IgG suppression in mice dosed with additional rapamycin-comprising synthetic nanocarriers, while IgG response suppression in mice treated with rapamycin-comprising synthetic nanocarriers only admixed with AAV was pronounced, but incomplete, especially after boost (FIG. 15B).

### Example 19: Additional Doses of Synthetic Nanocarriers Comprising an Immunosuppressant

Additional rapamycin-comprising synthetic nanocarriers was found to provide the highest potential for long-term AAV antibody suppression.

Although rapamycin-comprising synthetic nanocarrier co-administration with AAV and its further application were shown to effectively suppress antibodies to AAV, this suppression does not always reach the 100% level. Therefore, whether combining additional rapamycin-comprising synthetic nanocarrier injections at prime and follow-up administration of rapamycin-comprising synthetic nanocarriers would provide for a combined synergistic benefit was examined. Groups of 6-9 female C57BL/6 mice were injected on days 0 and 83 (i.v., tail vein) with 1×10¹⁰ VG of AAV-SEAP either alone or admixed with rapamycin, when comprised in synthetic nanocarriers, at 50 μg with one group additionally treated with rapamycin-comprising synthetic nanocarriers at 2 days prior to prime and boost (d-2 and d81), another was treated with two additional bi-weekly rapamycin-comprising synthetic nanocarrier injections both after prime and boost (d14, 28, 97 and 116) and the last one with a combination of those (d-2, 12, 28, 81, 97 and 116). IgG to AAV were measured as described in Example 14.

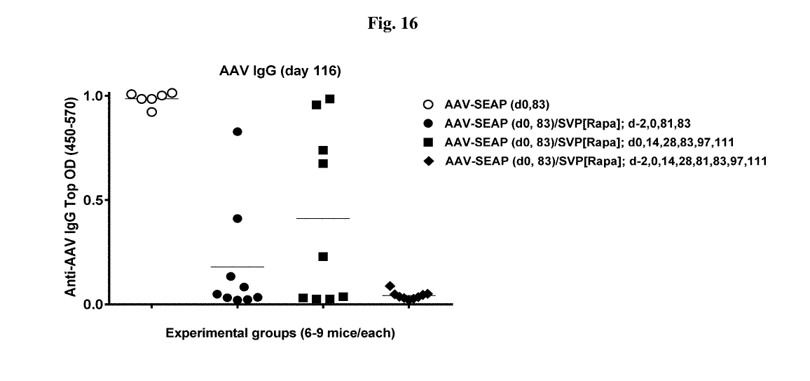

As shown before, administration of 50 μg of rapamycin, when comprised in synthetic nanocarriers, admixed to AAV-SEAP combined with pre-immunization rapamycin-comprising synthetic nanocarrier treatment (gr. 2; d-2, 0, 81, 83) led to profound AAV IgG suppression with no pre-boost conversions, as only 2 out of 9 mice showed detectable IgG levels (as determined by top OD) on day 90 (immediately after boost). Only 3 out of 9 (and only one of the three, strongly) were IgG-positive by day 116 (33 days post boost). In this study, follow-up (d14 and d28) treatments with rapamycin-comprising synthetic nanocarriers were as efficient pre-boost administration (no conversion). However, several mice started to convert post-boost (five out of nine; four of the five, strongly), becoming positive by day 116. Therefore, the combination of both rapamycin-comprising synthetic nanocarrier administration regimens was the most effective, as no conversions were observed up to day 116 (33 days post boost) (FIG. 16).

### Example 20: AAV-Driven Transgene Expression

It has been shown that, in a standard female mouse AAV transduction model, there is a benefit of co-administering of SVP[Rapa] with AAV, which results in higher transgene expression in vivo. This effect is further augmented by additional SVP[Rapa] administrations. In this example, it is demonstrated that a single SVP[Rapa] co-administration with AAV at prime and at boost improves transgene expression in a dose-dependent fashion and that this effect is, at least partially, inversely correlated with AAV antibody development. Furthermore, if a high dose of SVP[Rapa] capable of strongly elevating of AAV-driven transgene expression is evenly split in three parts, of which only one is co-administered with AAV and other two are administered separately prior to and after AAV injection, then the beneficial effect of SVP[Rapa] on transgene expression and SVP[Rapa]-mediated suppression of AAV antibody development are not compromised.

Specifically, four groups of 10 female C57BL/6 mice were injected (intravenously (i.v.), tail vein) with 1×10¹⁰ VG of AAV8-SEAP without or with SVP[Rapa]. The following doses of SVP[Rapa] were used: a single 50 μg dose (admixed and co-administered with AAV), a single 150 μg dose (admixed and co-administered with AAV), and a 150 μg dose, which was split in three 50 μg injections (one admixed and co-administered with AAV and two administered separately, at 2 days prior to AAV injection and at 2 days after AAV injection).

At time indicated (days 7, 12, 19, 47 and 75) mice were bled, and the serum was separated from whole blood and stored at −20±5° C. until analysis. Then, the IgG antibody to AAV was measured using an ELISA. Ninety-six-well plates were coated with the AAV overnight, washed and blocked on the following day and then diluted serum samples (1:40) were added to the plate and incubated. Following incubation, the plates were washed and goat anti-mouse IgG specific-HRP was added. The plates were incubated and washed again, and then the presence of IgG antibodies to AAV was detected by adding TMB substrate and measuring the signal at an absorbance of 450 nm with a reference wavelength of 570 nm. The intensity of the signal presented as top optical density, OD, is directly proportional to the quantity of IgG antibody in the sample.

Separately, secreted alkaline phosphatase (SEAP) levels in serum were measured using an assay kit from ThermoFisher Scientific (Waltham, Mass., USA). Briefly, sera samples and positive controls were diluted in dilution buffer, incubated at 65° C. for 30 min, then cooled to room temperature, plated into 96-well pates, and then incubated with assay buffer (5 min) and then substrate (20 min). Plates were then read on a luminometer at 477 nm.

Upon initial (post-prime) AAV IgG and SEAP detection and analysis, mice were rested, and then again bled on day 117 and boosted on day 125 with AAV-SEAP using the same AAV and SVP[Rapa] doses as at prime, i.e. the first group received no SVP[Rapa], and the following groups receiving 50 μg of SVP[Rapa] at boost, 150 μg of SVP[Rapa] at boost and 50 μg of SVP[Rapa] three times: 2 days prior to boost, at boost (admixed and co-administered with AAV), and 2 days after boost. Mice were then bled on days 132 and 138 (7 and 13 days post-boost) and SEAP serum levels were determined as specified above.

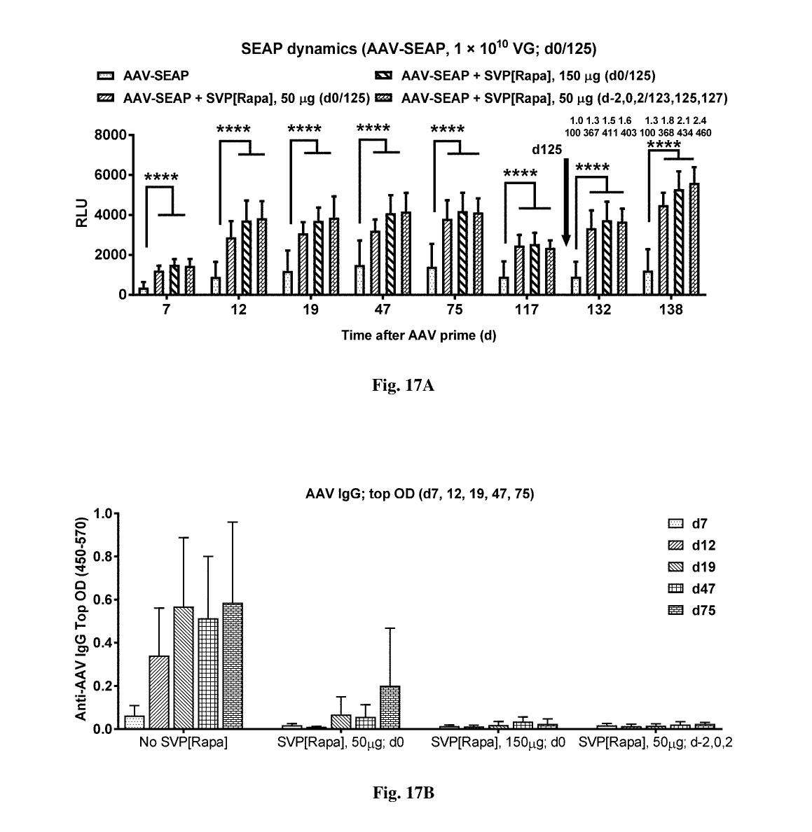

All groups treated with SVP[Rapa] showed increased SEAP levels immediately after the prime compared to those in untreated mice (FIG. 17A, gr. 1 vs. gr. 2-4), these differences were statistically significant (**** −p<0.0001) and persisted for several months. At most of the early time-points, SEAP levels in groups treated with 150 μg of SVP[Rapa] (as a single or a split dose; gr. 3 and 4) were higher than in the group treated with the lower, 50 μg dose (gr. 2), although in the long run (d75-117) all of these levels became equal. To a degree, this correlated with the early dynamics of AAV IgG development in the mice in groups treated with 150 μg of SVP[Rapa] showing no IgG conversions up to and including day 75 (FIG. 17B, gr. 3 and 4) while some mice in the group treated with the lower, 50 μg dose (FIG. 17B, gr. 2) demonstrated detectable antibody on day 19, with four out of ten (40%) converting by day 75 (FIG. 17B). Notably, all mice injected with AAV without SVP[Rapa] rapidly became AAV IgG-positive (FIG. 17B, gr. 1).

After the day 125 boost (shown by arrow in FIG. 17A), the difference between SVP[Rapa]-treated and untreated groups became even more profound (FIG. 17A, days 132 and 138). Notably, immediately after the boost (d132) there was no SEAP elevation in the mice that were not treated with SVP[Rapa] (ratios of post-boost SEAP expression to pre-boost expression on d117 are shown as a top line in FIG. 17A), while all SVP[Rapa]-treated groups showed immediate elevation (FIG. 17A, gr. 2-4, d132). Interestingly, SEAP levels in the untreated group and in the group treated with the low 50 μg dose of SVP[Rapa] progressed in a similar fashion to day 138 with their relative expression (shown as the lower line in FIG. 17A, levels in untreated gr. 1 assigned a number of ‘100’) staying the same (50 fig-treated group consistently having ˜3.5-fold higher SEAP). At the same time, SEAP levels in both groups of mice treated with the higher (150 μg) doses of SVP[Rapa] had further elevated transgene expression from day 132 to day 138, i.e. from being ˜4-fold higher to becoming ˜4.5-fold higher than in untreated mice, and at one instance, even became statistically different from that of mice treated with the lower (50 μg) dose of SVP[Rapa] (FIG. 17A, gr. 2 vs. gr. 4; day 138; p<0.05).

Therefore, AAV-driven transgene expression was found to be elevated by the co-administration of admixed SVP[Rapa] in a dose-dependent manner at both prime and boost. This effect inversely, although not completely, correlated with the suppression of antibodies to AAV but was not dependent on the SVP[Rapa] dose being delivered as a single dose admixed to AAV or as a split dose with some of it being admixed to AAV and some administered separately.

1. A method comprising:
coadministering synthetic nanocarriers comprising an immunosuppressant and a viral vector to a subject, and
administering at least one pre-dose and/or at least one post-dose of the synthetic nanocarriers comprising an immunosuppressant without the viral vector to the subject.

2. The method of claim 1, wherein at least one pre-dose and at least one post-dose is administered to the subject.

3. The method of claim 1, wherein at least two pre-doses are administered to the subject.

4. The method of claim 1, wherein at least two post-doses are administered to the subject.

5. The method of claim 1, wherein the coadministering is repeated in the subject.

6. The method of claim 5, wherein at least one pre-dose and/or at least one post-dose of the synthetic nanocarriers comprising an immunosuppressant without the viral vector is administered to the subject with each repeated coadministering step.

7. The method of claim 6, wherein at least one pre-dose and at least one post-dose is administered to the subject with each repeated coadministering step.

8. The method of claim 6, wherein at least two pre-doses are administered to the subject with each repeated coadministering step.

9. The method of claim 6, wherein at least two post-doses are administered to the subject with each repeated coadministering step.

10. The method of claim 1, wherein administration of the pre-dose(s) and/or post-dose(s) occurs within 1 month prior or subsequent to, respectively, a coadministration.

11. The method of claim 10, wherein administration of the pre-dose(s) and/or post-dose(s) occurs within 2 weeks prior or subsequent to, respectively, to a coadministration.

12. The method of claim 11, wherein administration of the pre-dose(s) and/or post-dose(s) occurs within 1 week prior or subsequent to, respectively, to a coadministration.

13. The method of claim 12, wherein administration of the pre-dose(s) and/or post-dose(s) occurs within 3 days prior or subsequent to, respectively, to a coadministration.

14. The method of claim 13, wherein administration of the pre-dose(s) and/or post-dose(s) occurs within 2 days prior or subsequent to, respectively, to a coadministration.

15. The method of claim 14, wherein administration of the pre-dose(s) and/or post-dose(s) occurs within 1 day prior or subsequent to, respectively, to a coadministration.

16. The method of claim 15, wherein administration of the pre-dose(s) and/or post-dose(s) occurs within 12 hours prior or subsequent to, respectively, to a coadministration.

17. The method of claim 16, wherein administration of the pre-dose(s) and/or post-dose(s) occurs within 6 hours prior or subsequent to, respectively, to a coadministration.

18. The method of claim 17, wherein administration of the pre-dose(s) and/or post-dose(s) occurs within 1 hour prior or subsequent to, respectively, to a coadministration.

19-26. (canceled)

27. A method comprising:
to a first subject, (1) coadministering (a) a dose of immunosuppressant comprised in synthetic nanocarriers and (b) a dose of a viral vector, and (2) administering, without a dose of the viral vector, (c) a pre-dose and/or a post-dose of the immunosuppressant comprised in synthetic nanocarriers,
wherein the amount of the immunosuppressant of (a) and (c) together is equal to an amount of immunosuppressant of (d) a dose of the immunosuppressant comprised in synthetic nanocarriers that when coadministered with the viral vector, without a pre-dose or a post-dose of the immunosuppressant coupled to synthetic nanocarriers, reduces an immune response against the viral vector or increases transgene expression of the viral vector in a second subject.

28-91. (canceled)

92. A kit comprising:
one or more pre-doses or one or more post-doses, each as described in any one of the preceding claims, and
a dose of synthetic nanocarriers comprising an immunosuppressant for coadministration with a viral vector.

93-98. (canceled)
